(19) United States
(12) Patent Application Publication
(10) Pub. No.: US 2004/0236092 A1
(43) Pub. Date: Nov. 25, 2004

(54) Peptidologlycan recognition protein encoding nucleic acids and methods of use thereof

(76) Inventors: Roman Dziarski, Chesterton, IN (US); Chao Liu, Seattle, WA (US); Zhaojun Xu, Tokyo (JP); Dipika Gupta, Chesterton, IN (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/483,651

(22) PCT Filed: Jul. 15, 2002
(86) PCT No.: PCT/US02/22428

Related U.S. Application Data
(60) Provisional application No. 60/305,049, filed on Jul. 13, 2001.

(51) Int. Cl. C07K 16/18; C07H 21/04

(52) U.S. Cl. 536/23.53; 530/388.1

(57) ABSTRACT

Novel human PGRP genes and their encoded proteins are provided herein. The peptidoglycan recognition proteins encoded by the disclosed nucleic acid sequences play a pivotal role in the innate immune response. PGRP genes and their encoded proteins provide valuable therapeutic targets for the design of agents which modulate the immune response to bacterial infection.

Pursuant to 35 U.S.C. §202(c) it is acknowledged that the U.S. Government has certain rights in the invention described herein, which was made in part with funds from the National Institutes of Health, USPHS Grant Number, AI2879.

### FIELD OF THE INVENTION

The present invention relates to the fields of medicine and molecular biology. More specifically, the invention provides novel nucleic acid molecules and proteins encoded thereby which may be used as agents to modulate the innate immune system.

### BACKGROUND OF THE INVENTION

Several publications and patent documents are referenced in this application in parentheses in order to more fully describe the state of the art to which this invention pertains. The disclosure of each of these publications is incorporated by reference herein.

Innate immunity is the first line of defense against microorganisms. It includes cellular components, which are primarily phagocytic and pro-inflammatory cells (neutrophils and macrophages in vertebrates) and humoral components, such as bacteriolytic enzymes (e.g., lysozyme), complement, mannose-binding protein, and soluble CD14 (1-3). The components of the innate immune system that discriminate between microorganisms and self are able to recognize conserved motifs found in microorganisms but not in higher eukaryotes. When present on cells, they are referred to as pattern recognition receptors. In mammals, pattern recognition receptors can induce phagocytosis (e.g., scavenger receptor, or mannan and β-glucan receptors), chemotaxis (e.g. N-formyl-methionine receptor), or secretion of pro-inflammatory mediators (e.g., CD14 and toll-like receptors (TLR; 1-3). Some mammalian pattern recognition receptors (e.g., CD14 or TLR2) recognize multiple microbial components (1-11), whereas others (e.g., TLR4 or TLR9) are more selective (1-3, 11-13). Innate immune mechanisms are highly conserved in evolution and are often similar in vertebrates and invertebrates. For example, both mammals and insects have highly conserved families of TLR receptors, although individual members of these families seem to have different functions in mammals and insects (1-3, 9-14).

Peptidoglycan (PGN) is an essential cell wall component of virtually all bacteria (15, 16) and, thus, it is an excellent target for recognition by the eukaryotic innate immune system. Indeed, PGN induces strong antibacterial responses in insects (17, 18) and activates monocytes, macrophages, and B lymphocytes in mammals (4, 5, 16, 19-21). Activation of mammalian monocytic cells by PGN is mediated by CD14 (4-8) and TLR2 (9-11), and leads to the production of numerous inflammatory mediators (4-6, 19, 20). These PGN-induced mediators can reproduce most of the major clinical manifestations of bacterial infections, including fever, inflammation, leukocytosis, hypotension, decreased peripheral perfusion, malaise, sleepiness, decreased appetite, and arthritis (5, 16).

One of the antimicrobial mechanisms in insects activated by PGN is the prophenoloxidase cascade (18). It is present in hemolymph and cuticle and can be initiated by binding of PGN to a 19-kDa protein, peptidoglycan recognition protein (PGRP; 22). PGRP from a moth (Trichoplusia ni) and a silkworm (Bombyx mori) have recently been cloned (23, 24). Moreover, mouse and human PGRP homologs have also been cloned (23), thus demonstrating that this protein has been highly conserved in evolution.

Mouse PGRP binds PGN with nanomolar affinity (25), and mouse and human PGRP are expressed in the bone marrow and neutrophils (23, 25). Mouse PGRP inhibits growth of Gram-positive bacteria and, therefore, it is likely to function as an antibacterial protein in neutrophils (25).

Recent data from the Drosophila melanogaster genome project have identified a family of 12 highly diversified PGRP homologs, distributed at 8 loci on 3 different chromosomes (26). Based on the predicted structures of the gene products, Drosophila PGRPs could be grouped into two classes: short PGRPs (PGRP-S), which are small extracellular proteins similar to the original PGRP, and long PGRPs (PGRP-L), which have long transcripts and are either intracellular or membrane-spanning proteins. Many of these Drosophila PGRPs are expressed in immune competent organs, such as fat body, gut, and hemocytes, and their expression is upregulated by injections of PGN (26).

Recently, insect PGRP-SA was shown to be required for effective immunity to Gram-positive bacteria and induction of anti-bacterial peptides in Drosophila (35), and PGRP-LC was shown to mediate production of anti-bacterial peptides in Drosophila in response to Gram-negative and Gram-positive bacteria (36, 37). Drosophila PGRP-LS also mediates phagocytosis of bacteria (38). Thus, insect PGRPs play an important role in innate immunity to bacteria. Because innate immunity is highly conserved from insects to mammals, PGRPs are also likely to play an important role in innate immunity in mammals.

### SUMMARY OF THE INVENTION

In view of the essential role played by the innate immune system in recognition and elimination of deleterious bacteria introduced via environmental exposure, there is a need to provide molecules which modulate this process. The identification of such molecules and the molecular elucidation of the role they play in innate immune defense mechanisms provide targets for novel efficacious anti-bacterial therapeutic agents.

Thus, in accordance with the present invention, novel, biological molecules useful for the identification, detection, and/or molecular characterization of components involved in an immune response to bacterial infection are disclosed. Also, provided are reagents useful for the development of anti-microbial therapeutic agents. Such anti-microbial agents have utility in treatment of patients suffering from systemic or localized bacterial infections or in prophylactic approaches for the treatment of such infections. Systemic bacterial infection (sepsis) is a major source of complications arising after surgical intervention and can be life threatening if not treated promptly and effectively.

According to one aspect of the invention, an isolated nucleic acid molecule is provided which includes a sequence encoding a PGRP of about 576 amino acids in length. The encoded protein, referred to herein as PGRP-L, comprises a multi-domain structure including an N-terminal signal peptide, two predicted transmembrane domains, and three contiguous PGRP domains located in the extracellular portion.

In a preferred embodiment of the invention, an isolated nucleic acid molecule is provided that encodes a human PGRP-L protein. In a particularly preferred embodiment, a human PGRP-L protein has an amino acid sequence the same as SEQ ID NO: 2. An exemplary PGRP-L nucleic acid molecule of the invention comprises SEQ ID NO: 1.

According to another aspect of the invention, a second isolated nucleic acid molecule is provided which includes a sequence encoding a PGRP of about 341 amino acids. The encoded protein, referred to herein as PGRP-Iα contains a multi-domain structure including an N-terminal signal peptide, two predicted transmembrane domains, and four PGRP domains, two of which are located individually on different extracellular portions and the remaining two are found on the cytoplasmic portion.

In another embodiment of the invention, an isolated nucleic acid molecule is provided that encodes a human PGRP-Iα protein. In a particularly preferred embodiment, a human PGRP-Iα protein has an amino acid sequence the same as SEQ ID NO: 4. An exemplary PGRP-Iα nucleic acid molecule of the invention comprises SEQ ID NO: 3.

According to yet another aspect of the invention, an isolated nucleic acid molecule is provided which includes a sequence encoding a protein of about 373 amino acids in length. The encoded protein, referred to herein as PGRP-Iβ, contains a multi-domain structure including an N-terminal signal peptide, two predicted transmembrane domains, and four PGRP domains, one of which is located on an extracellular portion and the remaining three are found on the cytoplasmic portion.

The invention also includes an isolated nucleic acid molecule that encodes a PGRP-Iβ protein. In a particularly preferred embodiment, a human PGRP-Iβ protein has an amino acid sequence the same as SEQ ID NO: 6. An exemplary PGRP-Iβ nucleic acid molecule of the invention comprises SEQ ID NO: 5.

According to another aspect of the present invention, an isolated nucleic acid molecule is provided, which has a sequence selected from the group consisting of: (1) SEQ ID NO: 1; (2) a sequence specifically hybridizing with preselected portions or all of the complementary strand of SEQ ID NO: 1 comprising nucleic acids encoding amino acids 1-576 of SEQ ID NO: 2; (3) a sequence encoding preselected portions of SEQ ID NO: 1 within nucleotides 1-1794, (4) SEQ ID NO: 3; (5) a sequence specifically hybridizing with preselected portions or all of the complementary strand of SEQ ID NO: 3 comprising nucleic acids encoding amino acids 1-341 of SEQ ID NO: 4; (6) a sequence encoding preselected portions of SEQ ID NO: 3 within nucleotides 1-1173, (7) SEQ ID NO: 5; (8) a sequence specifically hybridizing with preselected portions or all of the complementary strand of SEQ ID NO: 5 comprising nucleic acids encoding amino acids 1-373 of Sequence ID NO: 6; (9) a sequence encoding preselected portions of SEQ ID NO: 5 within nucleotides 1-1194; (10) a sequence comprising nucleotides 763-1459 of PGRP-L ORF (SEQ ID NO: 21); (11) a sequence comprising nucleotides −26 to 1459 of PGRP-L ORF (SEQ ID NO: 22); (12) a sequence comprising nucleotides 1136 of PGRP-L ORF through the poly-A tail (SEQ ID NO: 23); (13) a sequence comprising nucleotides 596-1019 of PGRP-Iα ORF (SEQ ID NO: 24).

Such partial sequences are useful as probes to identify and isolate homologues of the PGRP genes of the invention. Additionally, isolated nucleic acid sequences encoding natural allelic variants of the nucleic acids of SEQ ID NOS: 1, 3, and 5 are also contemplated to be within the scope of the present invention. The term natural allelic variants will be defined hereinbelow.

According to another aspect of the present invention, antibodies immunologically specific for part or all of the human PGRP proteins described hereinabove are provided.

Host cells comprising at least one of the PGRP encoding nucleic acids are also provided. Such host cells include but are not limited to bacterial cells, fungal cells, insect cells, mammalian cells, and plant cells. Host cells overexpressing one or more of the PGRP encoding nucleic acids of the invention provide valuable research tools for many applications, including, but not limited to, screening patients predisposed to bacterial infections and developing anti-microbial agents for therapeutic and prophylactic intervention. PGRP expressing cells also comprise a biological system useful in methods for identifying modulators of PGRPs.

Another embodiment of the present invention encompasses methods for screening cells expressing PGRP encoding nucleic acids for anti-microbial properties. Such methods provide medical researchers with data correlating expression of a particular PGRP gene with a particular anti-microbial resistant phenotype.

In another embodiment, the present invention encompasses methods for screening cells expressing PGRP encoding nucleic acids, wherein agents capable of modulating PGRP-mediated anti-microbial activity can be identified. The identification of such agents provides medical practitioners with valuable tools with which to treat patients suffering from bacterial infections.

Diagnostic methods are also encompassed by the present invention. Accordingly, suitable oligonucleotide probes are provided which hybridize to the nucleic acids of the invention. Such probes may be used to advantage in screening tissue samples derived from patients exhibiting symptoms consistent with innate immune response deficiencies for altered expression of particular PGRP genes. Once a tissue sample has been characterized as to the PGRP gene(s) expressed therein, modulators identified in the cell line screening methods described above may be administered to modulate anti-microbial activity.

Also provided are compositions comprising at least one of the PGRP molecules of the present invention in a pharmaceutically acceptable carrier. Such compositions comprising PGRP molecules may be administered to a patient in need thereof alone or in combination with other prophylactic and/or therapeutic agents, such as for example, antibiotics.

The methods of the invention may be applied to kits. An exemplary kit of the invention comprises PGRP gene specific oligonucleotide probes and/or primers, PGRP encoding DNA molecules for use as a positive control, buffers, and an instruction sheet. A kit for practicing the cell line screening method includes frozen cells comprising the PGRP genes of the invention, suitable culture media, buffers and an instruction sheet.

In a further aspect of the invention, transgenic knockout mice are disclosed. Mice may be generated in which at least one PGRP gene has been knocked out. Such mice provide a valuable biological system for assessing susceptibility to bacterial infections in an in vivo model.

Various terms relating to the biological molecules of the present invention are used hereinabove and also throughout the specification and claims. The terms “percent similarity” and “percent identity (identical)” are used as set forth in the UW GCG Sequence Analysis program (Devereux et al. NAR 12:387-397 (1984)).

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the genomic DNA of a prokaryote or eukaryote.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The nucleic acid molecules of the invention may be cloned and expressed in vectors. Such vectors may be in the form of, for example, a plasmid, a replication competent or defective virus or phage vector or a replicon provided typically with an origin of replication, optionally a promoter for the expression of the polynucleotide and optionally a regulator of the promoter. The vector may contain one or more selectable marker genes, for example an ampicillin resistance gene in the case of a bacterial plasmid or a neomycin resistance gene for a mammalian vector. The vector may be used in vitro, for example for the production of RNA or protein. The vector may be further used to transform, transfect, infect or transduce a host cell or an organism. The present invention further contemplates the use of host cells and organisms harboring or expressing the PGRP nucleic acid sequences or polypeptides of the invention for the identification of agents that affect the activity of the PGRP.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

Amino acid residues are identified in the present application according to the three-letter or one-letter abbreviations in the following Table:

TABLE 1

| | 3-letter | 1-letter |
|---|---|---|
| Amino Acid | Abbreviation | Abbreviation |
| L-Alanine | Ala | A |
| L-Arginine | Arg | R |
| L-Asparagine | Asn | N |
| L-Aspartic Acid | Asp | D |
| L-Cysteine | Cys | C |
| L-Glutamine | Gln | Q |
| L-Glutamic Acid | Glu | E |
| Glycine | Gly | G |
| L-Histidine | His | H |
| L-Isoleucine | Ile | I |
| L-Leucine | Leu | L |
| L-Methionine | Met | M |
| L-Phenylalanine | Phe | F |
| L-Proline | Pro | P |
| L-Serine | Ser | S |
| L-Threonine | Thr | T |
| L-Tryptophan | Trp | W |
| L-Tyrosine | Tyr | Y |
| L-Valine | Val | V |
| L-Lysine | Lys | K |

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like). With respect to antibodies of the invention, the term “immunologically specific” refers to antibodies that bind to one or more epitopes of a protein of interest (e.g., PGRP-L, PGRP-Iα, PGRP-Iβ), but which do not substantially recognize and bind other molecules in a sample containing a mixed population of antigenic biological molecules.

The present invention also includes active portions, fragments, derivatives and functional or non-functional mimetics of PGRP polypeptides or proteins of the invention. An “active portion” of PGRP means a peptide that is less than the full length PGRP, but which retains measurable biological activity.

A “fragment” or “portion” of PGRP means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the PGRP sequence, antigenic determinants, or epitopes are useful for eliciting immune responses to a portion of the PGRP amino acid sequence.

A “derivative” of PGRP or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of original the PGRP.

As mentioned above, the PGRP polypeptide or protein of the invention includes any analogue, fragment, derivative or mutant which is derived from a PGRP and which retains at least one property or other characteristic of PGRP. Different “variants” of PGRP exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the PGRP, (c) variants in which one or more amino acids include a substituent group, and (d) variants in which PGRP is fused with another peptide or polypeptide such as a fusion partner, a protein tag or other chemical moiety, that may confer useful properties to PGRP, such as, for example, an epitope for an antibody, a polyhistidine sequence, a biotin moiety and the like. Other PGRPs of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post-translational modification forms result in derivatives of PGRP that retain any of the biological properties of PGRP, they are included within the scope of this invention.

The term “functional” as used herein implies that the nucleic or amino acid sequence is functional for the recited assay or purpose.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

One common formula for calculating the stringency conditions required to achieve hybridization between nucleic acid molecules of a specified sequence homology (Sambrook et al., 1989):

Tₘ=81.5°φC.+16.6Log[Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such sequences would be considered substantially homologous to the nucleic acid sequences of the invention.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

“Mature protein” or “mature polypeptide” shall mean a polypeptide possessing the sequence of the polypeptide after any processing events that normally occur to the polypeptide during the course of its genesis, such as proteolytic processing from a polyprotein precursor. In designating the sequence or boundaries of a mature protein, the first amino acid of the mature protein sequence is designated as amino acid residue 1. As used herein, any amino acid residues associated with a mature protein not naturally found associated with that protein that precedes amino acid 1 are designated amino acid −1, −2, −3 and so on. For recombinant expression systems, a methionine initiator codon is often utilized for purposes of efficient translation.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other manners, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

An “immune response” signifies any reaction produced by an antigen, such as a viral antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured in various cellular (in vitro) or animal (in vivo) systems. Such immune responses may be important in protecting the host from disease and may be used prophylactically and therapeutically.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

The nucleic acids, proteins, antibodies, cell lines, methods, and kits of the present invention may be used to advantage to identify targets for the development of novel agents having anti-microbial properties. The transgenic mice of the invention may be used as an in vivo model system for deficiencies of the innate immune system.

The human PGRP molecules, methods, and kits described above may also be used as research tools to facilitate the elucidation of genotypes associated with a predisposition or enhanced susceptibility to microbial infection. Moreover, the human PGRP molecules described above, and modulators thereof, provide promising reagents for the prevention and/or treatment of bacterial infections and complications arising from the same.

### BRIEF DESCRIPTION OF THE DRAWINGS

FIG. 1 shows the genomic organization of four human PGRP genes. Exons coding for the proteins and intervening introns are shown.

FIG. 2 depicts the domain/structure and cellular location of human PGRP proteins.

FIG. 3 shows a phylogenetic tree of mammalian and insect PGRPs. Human PGRPs are in bold print. For branches supported by bootstrap analysis with the percentage of 1000 replications higher than 85%, the percentage is indicated. The bar indicates the p-distance. PGRP-S sequence is from ref. 23 (AF076483). The sequences of PGRP-L, PGRP-Iα, and PGRP-Iβ are available from GenBank under accession numbers AF384856, AY035376, and AY035377, respectively. Abbreviations: B. m., Bombyx mori; C. d., Camelus dromedarius; D. m., Drosophila melanogaster; H. s., Homo sapiens; M. m., Mus musculus; R. n., Rattus norvegicus; T. n., Trichoplusia ni. C. d. PGRP-S, AJ131676; R. n. PGRP-S, AF154114; M. m. PGRP-L, AF149837; M. m. PGRP-S, AF076482; B. m. PGRP-S, AB016249; T. n. PGRP-S, AF076481; D. m. PGRP-LAa1, AF313393; D. m. PGRP-LAb, AF207535; D. m. PGRP-LAc, AF207536; D. m. PGRP-LB, AF207537; D. m. PGRP-LC, AF207539; D. m. PGRP-LD, AF313389; D. m. PGRP-LE, AF313391; D. m. PGRP-SA, AF207541; D. m. PGRP-SClb, AF207542.

FIG. 4 shows the expression pattern of PGRP mRNA in 76 human tissues. Multiple Tissue Expression Arrays were hybridized with probes specific for the indicated PGRPs or ubiquitin and exposed to an X-ray film for 6 hrs (PGRP-S), 15 hrs (PGRP-L), 4 days (PGRP-Iα), 9 days (PGRP-Iβ), or 3 hrs (ubiquitin). A1, whole brain; B1, cerebral cortex; C1, frontal lobe; D1, parietal lobe; E1, occipital lobe; F1, temporal lobe; G1, p. g. of cerebral cortex; H1, pons; A2, left cerebellum; B2, right cerebellum; C2, corpus callosum; D2, amygdala; E2, caudate nucleus; F2, hippocampus; G2, medulla oblongata; H2, putamen; A3, substantia nigra; B3, accumbens nucleus; C3, thalamus; D3, pituitary gland; E3, spinal cord; A4, heart; B4, aorta; C4, left atrium; D4, right atrium; E4, left ventricle; F4, right ventricle; G4, interventricular septum; H4, apex of the heart; A5, esophagus; B5, stomach; C5, duodenum; D5, jejunum; E5, ileum; F5, ilocecum; G5, appendix; H5, ascending colon; A6, transverse colon; B6, descending colon; C6, rectum; A7, kidney; B7, skeletal muscle; C7, spleen; D7, thymus; E7, peripheral blood leukocytes; F7, lymph node; G7, bone marrow; H7, trachea; A8, lung; B8, placenta; C8, bladder; D8, uterus; E8, prostate; F8, testis; G8, ovary; A9, liver; B9, pancreas; C9, adrenal gland; D9, thyroid gland; E9, salivary gland; F9, mammary gland; A10, HL-60 leukemia; B10, S3 HeLa; C10, K-562 leukemia; D10, MOLT-4 leukemia; E10, Raji Burkitt's lymphoma; F10, Daudi Burkitt's lymphoma; G10, SW480 colorectal adenocarcinoma; H10, A549 lung carcinoma; A11, fetal brain; B11, fetal heart; C11, fetal kidney; D11, fetal liver; E11, fetal spleen; F11, fetal thymus; G11, fetal lung; A12, yeast RNA; B12, yeast tRNA; C12, E. coli rRNA; D12, E. coli DNA; E12, poly r(A); F12, human Cₒt-1 DNA; G12, 100 ng human DNA; H12, 500 ng human DNA. The following positions have no RNA or DNA: F3, G3, H3, D6, E6, F6, G6, H6, H8, G9, H9, H11.

FIG. 5 shows the expression pattern of PGRP mRNA on Northern blots and sizes of mRNA transcripts in the digestive and immune system. Multiple Tissue Northern blots were hybridized with the indicated probes and exposed to an X-ray film for: PGRP-L, 12 hrs; PGRP-S, 2 days (digestive) or 5 hrs (immune); PGRP-Iα, 19 hrs (digestive) or 3 days (immune); PGRP-Iβ, 2 days; P-actin, 2 hrs (digestive) or 30 min (immune). RNA size markers are shown on the left.

FIG. 6 reveals the expression of pattern of PGRP determined by PCR. PCR was performed on cDNA from the indicated 26 human tissues, and the PCR products were visualized on agarose gels by ethidium bromide staining (top panels) or on Southern blots by hybridization (lower panels).

FIG. 7 shows PGRP protein expression and binding assays to PGN and bacteria. Lysates of Cos-7 cells transfected with the indicated PGRPs or CD4 were incubated with Ni-NTA-agarose, control agarose, PGN-agarose, microgranular cellulose, Bacillus cells, or Micrococcus cells, as indicated, and washed three times (once for PGRP-Iβ lysates). Proteins eluted from the sediments were detected on Western blots with anti-V5 Abs. The results are from one of two similar experiments.

FIG. 8 shows the nucleic acid sequence (SEQ ID NO: 1) encoding the amino acid sequence of PGRP-L (SEQ ID NO: 2).

FIG. 9 shows the nucleic acid sequence (SEQ ID NO: 3) encoding the amino acid sequence of PGRP-Iα (SEQ ID NO: 4).

FIG. 10 shows the nucleic acid sequence (SEQ ID NO: 5) encoding the amino acid sequence of PGRP-IP (SEQ ID NO: 6).

FIG. 11 shows the nucleic acid sequences of SEQ ID Nos: 21, 22, 23, and 24.

### DETAILED DESCRIPTION OF THE INVENTION

The discovery of a PGRP family in Drosophila (26) suggested that a PGRP family might also exist in mammals. Indeed, as described herein, three novel homologs of human PGRP have been identified by searching the human genome. The cloning of their cDNAs, their differential expression in various tissues, and their ability to bind PGN and bacteria are reported herein. Other PGRPs have been previously identified, see for example PCT patent application No. WO 01/14545 A1, the entire disclosure of which is incorporated herein by reference. The pattern recognition molecules of the present invention may be used to advantage to modulate innate immunity in humans. They may also be used in the identification and development of prophylactic and/or therapeutic anti-microbial agents.

I. Preparation of PGRP-Encoding Nucleic Acid Molecules, PGRP Proteins, and Antibodies Thereto

A. Nucleic Acid Molecules

Nucleic acid molecules encoding the PGRP proteins of the invention may be prepared by two general methods: (1) synthesis from appropriate nucleotide triphosphates, or (2) isolation from biological sources. Both methods utilize protocols well known in the art. The availability of nucleotide sequence information, such as cDNAs having SEQ ID NOS: 1, 3, or 5 enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 5 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 5 kb double-stranded molecule. A synthetic DNA molecule so constructed may then be cloned and amplified in an appropriate vector.

Nucleic acid sequences encoding the PGRPs of the invention may be isolated from appropriate biological sources using methods known in the art. In a preferred embodiment, a cDNA clone is isolated from a cDNA expression library of human origin. In an alternative embodiment, utilizing the sequence information provided by the cDNA sequence, human genomic clones encoding PGRPs may be isolated. Alternatively, cDNA or genomic clones having homology with PGRP-L, PGRP-Iα, or PGRP-Iβ may be isolated from other species using oligonucleotide probes corresponding to predetermined sequences within the PGRP encoding nucleic acids.

In accordance with the present invention, nucleic acids having the appropriate level of sequence homology with the protein coding region of SEQ ID NOS: 1, 3, and 5 may be identified by using hybridization and washing conditions of appropriate stringency. For example, hybridizations may be performed, according to the method of Sambrook et al., (supra) using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° in 1×SSC and 1% SDS, changing the solution every 30 minutes.

Nucleic acids of the present invention may be maintained as DNA in any convenient cloning vector. In a preferred embodiment, clones are maintained in a plasmid cloning/expression vector, such as pT-Adv (Clontech, Palo Alto, Calif.), which is propagated in a suitable E. coli host cell.

PGRP-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of the cDNA having SEQ ID NO: 1. Such oligonucleotides are useful as probes for detecting or isolating PGRP genes. Antisense nucleic acid molecules may be targeted to translation initiation sites and/or splice sites to inhibit the translation of the PGRP-encoding nucleic acids of the invention. Such antisense molecules are typically between 15 and 30 nucleotides and length and often span the translational start site of PGRP encoding mRNA molecules.

It will be appreciated by persons skilled in the art that variants of these sequences exist in the human population, and must be taken into account when designing and/or utilizing oligos of the invention. Accordingly, it is within the scope of the present invention to encompass such variants, with respect to the PGRP sequences disclosed herein or the oligos targeted to specific locations on the respective genes or RNA transcripts. These variants may possess one or more changes, each of which may include one or more additions, deletions, or substitutions of amino acid residues. Preferably, the changes will not affect, or substantially affect, the structure of useful properties of the polypeptide. Thus, variants may suitably possess functional PGRP activity such as those described herein, or they may be poorly functional or inactive, yet contain substantially the secondary and tertiary structure of the native protein. Such PGRP molecules may be used to advantage to identify agents that specifically bind to or otherwise affect the PGRP activity. PGRP variants can be either naturally occurring (i.e., purified or isolated from a natural source) or synthetic (i.e., generated by biological expression of DNA that has been subjected to site-directed mutagenesis or produced by chemical synthetic techniques well known in the art). With respect to the inclusion of such naturally occurring variants, the term “natural allelic variants” is used herein to refer to various specific nucleotide sequences and variants thereof that would occur in a human population. The usage of different wobble codons and genetic polymorphisms which give rise to conservative or neutral amino acid substitutions in the encoded protein are examples of such variants. Additionally, the term “substantially complementary” refers to oligo sequences that may not be perfectly matched to a target sequence, but the mismatches do not materially affect the ability of the oligo to hybridize with its target sequence under the conditions described.

B. Proteins

Full-length PGRP-L, PGRP-Iα, and PGRP-Iβ proteins of the present invention may be prepared in a variety of ways, according to known methods. The proteins may be purified from appropriate sources, e.g., transformed bacterial or animal cultured cells or tissues, by immunoaffinity purification. However, this is not a preferred method due to the low levels of protein likely to be present in a given cell type at any time. The availability of nucleic acid molecules encoding PGRP proteins enables production of the proteins using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or Gibco-BRL, Gaithersburg, Md.

Alternatively, according to a preferred embodiment, larger quantities of PGRPs may be produced by expression in a suitable prokaryotic or eukaryotic system. For example, part or all of a DNA molecule, such as a cDNA having SEQ ID NO: 1, 3, or 5 may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

The human PGRP proteins produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

The human PGRPs of the invention, prepared by the aforementioned methods, may be analyzed according to standard procedures. For example, such proteins may be subjected to amino acid sequence analysis, according to known methods.

The present invention also provides antibodies capable of immunospecifically binding to proteins of the invention. Polyclonal antibodies directed toward human PGRPs may be prepared according to standard methods. In a preferred embodiment, monoclonal antibodies are prepared, which react immunospecifically with various epitopes of the PGRPs described herein. Monoclonal antibodies may be prepared according to general methods of Kohler and Milstein, following standard protocols. Polyclonal or monoclonal antibodies that immunospecifically interact with PGRPs can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules. Other uses of anti-PGRP antibodies are described below.

II. Uses of PGRP-Encoding Nucleic Acids, PGRPs and Antibodies Thereto

Innate immune responses which depend, in large part, on the activity of pattern recognition molecules, comprise a first line of defense against bacterial infection. Since PGRPs recognize peptidoglycans, an essential cell wall component of virtually all bacteria, the identification of novel human PGRPs and modulators thereto provides useful diagnostic and therapeutic tools for medical practitioners. PGRP molecules may be used to advantage to treat a patient in need thereof to effect modulation of an immune response. Modulators of PGRP activity may also be used to treat such patients.

Additionally, PGRP nucleic acids, proteins and antibodies thereto, according to this invention, may be used as research tools to identify other proteins that are intimately involved in the regulation of anti-microbial processes. Biochemical elucidation of molecular mechanisms which govern such processes facilitates the development of novel anti-microbial agents that may be used alone, or in conjunction with other anti-microbial agents (such as, for example, antibiotics), to control localized and/or systemic bacterial infections. Moreover, PGRP nucleic acids, proteins and antibodies thereto, may be useful in the development of therapeutic agents that modulate potentially life-threatening physiological responses (for example, an excessive, prolonged fever) that can occur in reaction to serious bacterial infections.

A. PGRP-Encoding Nucleic Acids

PGRP-encoding nucleic acids may be used for a variety of purposes in accordance with the present invention. PGRP-encoding DNA, RNA, or fragments thereof may be used as probes to detect the presence of and/or expression of genes encoding PGRPs. Methods in which PGRP-encoding nucleic acids may be utilized as probes for such assays include, but are not limited to: (1) in situ hybridization; (2) Southern hybridization; (3) northern hybridization; and (4) assorted amplification reactions such as polymerase chain reactions (PCR).

The PGRP-encoding nucleic acids of the invention may also be utilized as probes to identify related genes from other animal species. As is well known in the art, hybridization stringencies may be adjusted to allow hybridization of nucleic acid probes with complementary sequences of varying degrees of homology. Thus, PGRP-encoding nucleic acids may be used to advantage to identify and characterize other genes of varying degrees of relation to the PGRP genes of the invention. Such information enables further characterization of anti-microbial molecules which contribute to the innate immune response to bacteria. Additionally, they may be used to identify genes encoding proteins that interact with PGRP proteins (e.g., by the “interaction trap” technique), which should further accelerate identification of the components involved in the innate immune response. The PGRP encoding nucleic acids may also be used to generate primer sets suitable for PCR amplification of target PGRP DNA. Criteria for selecting suitable primers are well known to those of ordinary skill in the art.

Nucleic acid molecules, or fragments thereof, encoding PGRP genes may also be utilized to control the production of PGRP proteins, thereby regulating the amount of protein available to participate in anti-microbial responses. As mentioned above, antisense oligonucleotides corresponding to essential processing sites in PGRP-encoding mRNA molecules may be utilized to inhibit PGRP production in targeted cells. Alterations in the physiological amount of PGRPs may dramatically affect the ability of these proteins to serve as components of an anti-microbial response.

Host cells comprising at least one PGRP encoding DNA molecule are encompassed in the present invention. Host cells contemplated for use in the present invention include but are not limited to bacterial cells, fungal cells, insect cells, mammalian cells, and plant cells. The PGRP encoding DNA molecules may be introduced singly into such host cells or in combination to assess the phenotype of cells conferred by such expression. Methods for introducing DNA molecules are also well known to those of ordinary skill in the art. Such methods are set forth in Ausubel et al. eds., Current Protocols in Molecular Biology, John Wiley & Sons, NY, N.Y. 1995, the disclosure of which is incorporated by reference herein.

The availability of PGRP encoding nucleic acids enables the production of laboratory mice strains carrying part or all of the PGRP genes or mutated sequences thereof. Such mice may provide an in vivo model for development of novel anti-microbial agents. Alternatively, the PGRP nucleic acid sequence information provided herein enables the production of knockout mice in which the endogenous genes encoding PGRP-L, PGRP-Iα, or PGRP-Iβ have been specifically inactivated. Methods of introducing transgenes in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo.

The alterations to the PGRP gene envisioned herein include modifications, deletions, and substitutions. Modifications and deletions render the naturally occurring gene nonfunctional, producing a “knock out” animal. Substitutions of the naturally occurring gene for a gene from a second species results in an animal which produces a PGRP gene from the second species. Substitution of the naturally occurring gene for a gene having a mutation results in an animal with a mutated PGRP. A transgenic mouse carrying the human PGRP gene is generated by direct replacement of the mouse PGRP gene with the human gene. These transgenic animals are valuable for use in vivo assays for elucidation of other medical disorders associated with cellular activities modulated by PGRP genes. A transgenic animal carrying a “knock out” of a PGRP encoding nucleic acid is useful for the establishment of a nonhuman model for anti-bacterial activity involving PGRP regulation.

As a means to define the role that a PGRP plays in mammalian systems, mice may be generated that cannot make a particular PGRP because of a targeted mutational disruption of a PGRP gene.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring in fact, possess some or all of that alteration or genetic information, then they, too, are transgenic animals.

The alteration of genetic information may be foreign to the species of animal to which the recipient belongs, or foreign only to the particular individual recipient, or may be genetic information already possessed by the recipient. In the last case, the altered or introduced gene may be expressed differently than the native gene.

The altered PGRP gene generally should not fully encode the same PGRP protein native to the host animal and its expression product should be altered to a minor or great degree, or absent altogether. However, it is conceivable that a more modestly modified PGRP gene will fall within the compass of the present invention if it is a specific alteration.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro. Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated PGRP genes to selectively inactivate the wild-type gene in totipotent ES cells (such as those described above) and then generate transgenic mice. The use of gene-targeted ES cells in the generation of gene-targeted transgenic mice is known in the art.

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodouracil, (FIAU). By this counter selection, the number of homologous recombinants in the surviving transformants can be increased.

As used herein, a “targeted gene” or “knock-out” is a DNA sequence introduced into the germline or a non-human animal by way of human intervention, including but not limited to, the methods described herein. The targeted genes of the invention include DNA sequences which are designed to specifically alter cognate endogenous alleles.

Methods of use for the transgenic mice of the invention are also provided herein. Knockout mice of the invention can be injected with bacterial cells or treated with agents, such as cytokines, that are normally produced in response to bacterial infection. Such mice provide a biological system for assessing anti-bacterial properties as modulated by a PGRP gene of the invention. Accordingly, therapeutic agents which modulate the action of these recognition proteins, thereby altering the innate immune response to bacterial infection may be screened in studies using PGRP knock out mice.

As described above, PGRP-encoding nucleic acids are also used to advantage to produce large quantities of substantially pure PGRPs, or selected portions thereof.

B. PGRP Proteins and Antibodies

Purified full length PGRPs, or fragments thereof, may be used to produce polyclonal or monoclonal antibodies which also may serve as sensitive detection reagents for the presence and accumulation of PGRPs (or complexes containing PGRPs) in, for example, mammalian cells. Recombinant techniques enable expression of fusion proteins containing part or all of PGRPs. The full length proteins or fragments of the proteins may be used to advantage to generate an array of monoclonal antibodies specific for various epitopes of PGRPs, thereby providing even greater sensitivity for detection of PGRPs in cells.

Polyclonal or monoclonal antibodies immunologically specific for PGRPs may be used in a variety of assays designed to detect and quantitate the proteins. Such assays include, but are not limited to: (1) flow cytometric analysis; (2) immunochemical localization of PGRPs in cells; and (3) immunoblot analysis (e.g., dot blot, Western blot) of extracts from various cells. Additionally, as described above, anti-PGRP antibodies may be used for purification of PGRPs and any associated subunits (e.g., affinity column purification, immunoprecipitation).

From the foregoing discussion, it can be seen that PGRP-encoding nucleic acids, PGRP expressing vectors, PGRPs and anti-PGRP antibodies of the invention may be used to detect PGRP gene expression and/or alter PGRP accumulation for purposes of assessing the genetic and protein interactions involved in the development of anti-bacterial responses. It should be evident from the foregoing that reagents of the present invention may be used to modulate anti-bacterial responses, both to promote beneficial aspects of such responses and abrogate deleterious complications associated with such responses.

C. Methods and Kits Employing the Compositions of the Present Invention

Exemplary methods for detecting PGRP nucleic acid or polypeptides/proteins include:

a) comparing the sequence of nucleic acid in the sample with the PGRP nucleic acid sequence to determine whether the sample from the patient contains mutations; or

b) determining the presence, in a sample from a patient, of the polypeptide encoded by the PGRP gene and, if present, determining whether the polypeptide is full length, and/or is mutated, and/or is expressed at the normal level; or

c) using DNA restriction mapping to compare the restriction pattern produced when a restriction enzyme cuts a sample of nucleic acid from the patient with the restriction pattern obtained from normal PGRP gene or from known mutations thereof; or,

d) using a specific binding member capable of binding to a PGRP nucleic acid sequence (either normal sequence or known mutated sequence), the specific binding member comprising nucleic acid which hybridizes with the PGRP sequence, or substances comprising an antibody domain with specificity for a native or mutated PGRP nucleic acid sequence or the polypeptide encoded by it, the specific binding member being labelled so that binding of the specific binding member to its binding partner is detectable; or,

e) using PCR involving one or more primers based on normal or mutated PGRP gene sequence to screen for normal or mutant PGRP gene in a sample from a patient.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

In most embodiments for screening to identify/detect alleles giving rise to deficiencies in a patient's innate immune response to bacteria, a PGRP nucleic acid in a biological sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art. See U.S. Pat. Nos. 6,251,601, 6,255,456, 6,248,535, 6,248,521, 6,245,507, 6,245,297, and 6,238,868, each incorporated herein by reference.

The identification of a PGRP gene and its association with a particular innate immune deficiency paves the way for aspects of the present invention to provide the use of materials and methods, such as are disclosed and discussed above, for establishing the presence or absence in a test sample of a variant form of the gene, in particular an allele or variant specifically associated with an innate immune deficiency. There are numerous immunodeficiencies that manifest themselves clinically as inadequate immunity against microbial infections and for which the genetic defects responsible are not yet known (32). The compositions and methods of the present invention will facilitate screening of such immunodeficient patients for genetic alterations that could result in the production of altered levels of PGRPs or PGRPs having altered function. This may be done to anticipate the utility of administering an agent or agents which compensate for an altered PGRP activity associated with a variant form of a PGRP gene.

In still further embodiments, the present invention concerns immunodetection methods for binding, purifying, removing, quantifying or otherwise generally detecting biological components. The encoded proteins or peptides of the present invention may be employed to detect antibodies having reactivity therewith, or, alternatively, antibodies prepared in accordance with the present invention, may be employed to detect the encoded proteins or peptides. The steps of various useful immunodetection methods have been described in the scientific literature, such as, e.g., Nakamura et al. (1987).

In general, the immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing a PGRP gene encoded protein, peptide or a corresponding antibody, and contact the sample with an antibody or encoded protein or peptide, as the case may be, and then detect or quantify the amount of immune complexes formed under the specific conditions.

In terms of antigen detection, the biological sample analyzed may be any sample that is suspected of containing the PGRP antigen, such as a tissue section or specimen, a homogenized tissue extract, an isolated cell, a cell membrane preparation, separated or purified forms of any of the above protein-containing compositions.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. See U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a secondary antibody or a biotin/avidin ligand binding arrangement, as is known in the art.

In one broad aspect, the present invention encompasses kits for use in detecting expression of PGRP encoding nucleic acids in biological samples, including tissue or biopsy samples. Such a kit may comprise one or more pairs of primers for amplifying nucleic acids corresponding to a PGRP gene. The kit may further comprise samples of total mRNA derived from tissues expressing at least one or a subset of the PGRP genes of the invention, to be used as controls. The kit may also comprise buffers, nucleotide bases, and other compositions to be used in hybridization and/or amplification reactions. Each solution or composition may be contained in a vial or bottle and all vials held in close confinement in a box for commercial sale. In a further embodiment, the invention encompasses a kit for use in detecting PGRPs in cells derived from patients with innate immune response deficiencies comprising antibodies specific for PGRPs encoded by the PGRP nucleic acids of the present invention.

Another aspect of the present invention comprises screening methods employing host cells expressing one or more PGRP genes of the invention. An advantage of having discovered the complete coding sequences of PGRP-L, PGRP-Iα, or PGRP-Iβ is that cell lines that overexpress PGRP-L, PGRP-Iα, or PGRP-Iβ can be generated using standard transfection protocols. Cells transfected with a PGRP cDNA, which consequently express the corresponding PGRP as either a transmembrane protein or a secreted protein (whether a native or an engineered secreted isoform), provide an ideal system in which to analyze the biological activity of the PGRP. The overexpressing cell lines may be useful for a variety of applications: 1) Overexpressing cell lines may be used to delineate portions of expressed PGRPs that activate beneficial pathways/components of the innate immune response, but fail to activate pathways/components that contribute to adverse effects of the innate immune response. Such PGRP portions or fragments may be used as therapeutic agents in the treatment of patients with bacterial infections; 2) Overexpressing cell lines may be used to screen a plurality of agents to identify agents that modulate the ability of the expressed PGRP(s) to bind PGN and/or intact bacteria. Agents identified that are shown to enhance the ability of a PGRP(s) to bind the above ligands are of great clinical interest in that they may augment the activity of antibiotics and/or other anti-microbial drugs, thereby increasing their effectiveness. Agents identified that are shown to inhibit the ability of a PGRP(s) to bind the above ligands are also of great clinical interest in that they may abrogate or prevent some of the deleterious physiological consequences of prolonged activation of innate immune responses, thereby improving the short and long term prognosis of a patient; and 3) Overexpressing cell lines may be used to assess the ability of a PGRP(s) to bind strains of bacteria which have acquired an antibiotic resistant phenotype.

III. Preparation of Peptide Analogs

A peptide analog of the present invention can be made by exclusively solid phase techniques, by partial solid-phase techniques, by fragment condensation, by classical solution coupling, or, as long as the analog consists of only amino acids among the twenty naturally occurring amino acids corresponding to codons of the genetic code, by employing recombinant DNA techniques. Suitable host organisms for this purpose include, without limitation, E. coli, B. subtilis, S. cerevisiae, S. pombe and P. pastoris. Alternatively, insect or mammalian cells may be utilized.

Methods of making a polypeptide of known sequence by recombinant DNA techniques are well-known in the art. See, e.g., U.S. Pat. No. 4,689,318, which is incorporated herein by reference.

Methods for chemical synthesis of polypeptides are also well-known in the art and, in this regard, reference is made, by way of illustration, to the following literature: Yamashino and Li, J Am Chem Soc 100:5174-5178, 1978; Stewart and Young, Solid Phase Peptide Synthesis (WH Freeman and Co. 1969); Brown et al., JCS Peritin I, 1983, 1161-1167; M. Bodanszky et al., Bioorg Chem 2:354-362, 1973; U.S. Pat. Nos. 4,689,318; 4,632,211; 4,237,046; 4,105,603; 3,842,067; and 3,862,925, all of which are incorporated herein by reference.

IV. Administration of Peptide Analogs

The peptide analogs as described herein will generally be administered to a patient as a pharmaceutical preparation. The term “patient” as used herein refers to human or animal subjects. These protein analogs may be employed therapeutically, under the guidance of a physician for the treatment of bacterial infections.

The dose and dosage regimen of an analog of the present invention that is suitable for administration to a particular patient may be determined by a physician, in view of, for example, the patient's age, sex, weight, general medical condition, and the specific condition and severity thereof for which the peptide analog is being administered. The physician may also consider the route of administration of the peptide analog, the pharmaceutical carrier with which the peptide analog may be combined, and the peptide analog's biological activity.

Selection of a suitable pharmaceutical preparation depends upon the method of administration chosen. For example, the peptide analogs of the invention may be administered to treat patients with bacterial infections by direct injection into regions of the body in which a bacterial infection is found. Bacterial infections of the central nervous system (CNS), for example, are resistant to many forms of therapy, and as such, are good targets for localized treatment with peptide analogs of the present invention. For treatment of bacterial infections of the CNS, a pharmaceutical composition comprises the peptide analog dispersed in a medium that is compatible with cerebrospinal fluid. In a preferred embodiment, artificial cerebrospinal fluid (148 mM NaCl, 2.9 mM KCl. 1.6 mM MgCl₂, 6 H₂O, 1.7 mM CaCl₂, 2.2 mM dextrose) is utilized and the peptide analog is provided to neuronal tissue by intraventricular injection or by direct injection into the cerebrospinal fluid. In alternative embodiments, the pharmaceutical compositions may be administered by direct injection into any organ or body region (e.g., the peritoneal cavity).

Peptide analogs may also be administered parenterally by intravenous injection into the blood stream, or by subcutaneous, intramuscular or intraperitoneal injection. Pharmaceutical preparations for parenteral injection are known in the art. If parenteral injection is selected as a method for administering the peptide analogs, steps must be taken to ensure that sufficient amounts of the molecules reach their target cells to exert a biological effect. For example, when brain tissues are targeted, the lipophilicity of the peptide analogs, or the pharmaceutical preparation in which they are delivered may have to be increased so that the molecules can cross the blood-brain barrier to arrive at their target locations. Furthermore, the peptide analogs will have to be delivered in a cell-targeting carrier so that sufficient numbers of molecules will reach the target cells. Methods for increasing the lipophilicity of a molecule are known in the art.

The peptide analogs of the invention, or a pharmaceutically acceptable salt thereof, can be combined, over a wide concentration range (e.g., 0.001 to 11.0 wt %) with any standard pharmaceutically acceptable carrier (e.g., physiological saline, THAM solution, or the like) to facilitate administration by any of various routes including intravenous, subcutaneous, intramuscular, oral, intranasal, or inhalation.

Pharmaceutically acceptable salts of the peptide analogs of the invention can be prepared with any of a variety of inorganic or organic acids, such as for example, sulfuric, phosphoric, hydrochloric, hydrobromic, nitric, citric, succinic, acetic, benzoic and ascorbic. The peptide analogs can, for example, be advantageously converted to the acetate salt by dissolution in an aqueous acetic acid solution (e.g., 10% solution) followed by lyophilization.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the peptide or peptide analogs in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed. The pharmaceutical compositions will generally contain dosage units, e.g., tablet, capsule, powder, injection, teaspoonful and the like, from about 0.001 to about 10 mg/kg, and preferably from about 0.01 to about 0.1 mg/kg of the active ingredient.

The following examples are provided to illustrate various embodiments of the invention. They are not intended to limit the invention in any way.

### EXAMPLE I

### Isolation of PGRP cDNA

The following protocols are provided to facilitate the practice of the present invention.

### Experimental Procedures

Cloning of PGRP-L, PGRP-Iα, and PGRP-Iβ

Genes encoding PGRP-L, PGRP-Iα, and PGRP-Iβ were identified by searching GenBank databases for mammalian homologs of human PGRP-S using TBLASTN. A PGRP-L gene was found on chromosome 19, between nt 108,186 and 118,938 in clone CTB-187L3 (AC011492). The nucleotide sequence of PGRP-L had 74% identity with the unpublished cDNA for a mouse protein of unknown function deposited in GenBank under the name TAGL-α (AF149837). Based on this high homology to mouse TAGL-α (mouse PGRP-L), and on the sequences of three overlapping human EST clones (AV655895, AV719476, and BE672960, representing nt 787-1462, 1155-1731, and 1426-1731, respectively) comprising a PGRP-L open reading frame (ORF), five putative exons coding for human PGRP-L were identified. Based on this putative sequence, primers were designed for use in PCR (in a GeneAmp 9600 thermocycler, Perkin Elmer, Norwalk, Conn.) to amplify a 697 bp fragment (clone L11; SEQ ID NO: 21) from human universal cDNA (first strand cDNA synthesized from poly-A⁺ RNA pooled from 37 human tissues, Clontech, Palo Alto, Calif.). Clone L11 spanned from nt 763 to 1459 of a 1731 base pair (bp) long PGRP-L ORF sense primer; 5′ CCT CGG ACC TTT ACG CTT TTG GAC 3′ (SEQ ID NO: 7) and antisense primer; 5′ TGT AGT TGC CCA CTA TGG CCA CGC 3′ (SEQ ID NO: 8). Using this fragment, it was determined that PGRP-L was highly expressed in the liver (see below). Human liver cDNA (Clontech) was then used as source material for the PCR method to amplify a 1485 bp fragment (clone L62; SEQ ID NO: 22) covering 84% of PGRP-L ORF [exons 1 through 4, nt −26 through 1459; sense primer, 5′ CTT GGA AGC TGG AAT CCT GCA ACA 3′ (SEQ ID NO: 9) and antisense primer, 5′ TGT AGT TGC CCA CTA TGG CCA CGC 3′ (SEQ ID NO: 10). Both PCR fragments were ligated into the pT-Adv vector (Clontech). The L11 fragment was used as a probe to screen a bacteriophage λTriplEx human liver cDNA library (Clontech). Four partial PGRP-L clones (599 to 770 bp long; SEQ ID NO: 23) were identified, which overlapped with clone L62 and coded for exons 3, 4, and 5, and the untranslated sequence from the stop codon to the poly-A+ tail. To obtain full-length PGRP-L cDNA, clone L62 (nt −26 through 1459; SEQ ID NO: 22) was fused with one of the clones (nt 1137 through the poly-A tail) obtained from screening the λTriplEx liver cDNA library, by cutting both clones with SmaI at position 1426 and with SacI in the multiple cloning site of both vectors, and then re-ligating the two PGRP-L fragments with T4 DNA ligase (27). The cloned PGRP-L sequence was identical to the genomic sequence in clone CTB-187L3 (AC011492). The exon/intron junctions were also identical in all the clones and the three EST clones (AV655895, AV719476, and BE672960).

Nine exons were identified on human chromosome 1, region q21, of the 236c22 BAC clone (AC011666; located between nt 37,034 and 54,835) which encode PGRP-Iβ. The coding sequence of PGRP-Iβ was highly homologous to the unpublished cDNA of H. sapiens hypothetical protein SBBI67 (AF242518). In the same 236c22 BAC clone, eight putative exons coding for another highly homologous protein, PGRP-Iα, were identified between nt 74,880 and 87,746. Based on the sequences of the putative exons coding for PGRP-Iα and PGRP-Iβ, and the putative adjacent 5′ and 3′ untranslated regions, oligonucleotide primers were designed for use in PCR amplifications to clone full-length cDNAs encoding PGRP-Iα and PGRP-Iβ from human universal cDNA (first strand cDNA synthesized from poly-A⁺ RNA pooled from 37 human tissues; Clontech). The primers designed to be specific for each PGRP were as follows: for PGRP-Iα, sense, 5′ CCT CTC TTC CAG GGC TGC CGT C 3′ (SEQ ID NO: 11) and antisense, 5′ AGG GGG ACA CAA GGT GCT GAG C 3′ (SEQ ID NO: 12); and for PGRP-Iβ, sense, 5′ ACA GGA CCC ACA GAT ATC TGC TGC CAT C 3′ (SEQ ID NO: 13) and antisense, 5′ GCT TCT CTC AGT GTT TGA AAT GAG GCC AG 3′ (SEQ ID NO: 14). The PCR products were ligated into the pT-Adv vector (Clontech) and clones with the proper full-length PGRP-Iα and PGRP-Iβ inserts were selected and identified by restriction digestion and confirmed by sequencing. The sequencing revealed that in PGRP-Iα, putative exon 2, which was homologous to a similar exon in PGRP-Iβ, was not expressed. The differential expression of this exon accounted for the smaller size of PGRP-Iα, relative to that of PGRP-Iβ (341 vs 373 amino acids). In PGRP-Iβ, the first exon comprised part of the untranslated 5′ sequence and eight exons encoded the translated protein. The ORF of PGRP-Iβ was 12 bp longer than the ORF in SBBI67 (AF242518). The cloned PGRP-α and PGRP-Iβ sequences were 100% identical to the genomic sequences in BAC 236c22 (AC011492), except for a difference of two nucleotides (G275 and C967) in the ORF of PGRP-Iβ.

PGRP-S cDNA was cloned from human bone marrow cDNA (Clontech) by PCR amplification with sense 5′ CAC CAT GTC CCG CCG CTC TAT G 3′ (SEQ ID NO:15) and antisense 5′ GGG GGA GCG GTA GTG TGG CCA A 3′ (SEQ ID NO: 16) primers, designed based on published sequences (23; AF076483). The PCR products were ligated into the pcDNA3.1 mammalian expression vector (InVitrogen, Carlsbad, Calif.). The PGRP-S sequence was identical to the published cDNA and genomic sequences (AF076483 and AC007785).

Sequence Analysis

DNA sequencing was performed using an ABI Prism 377XL automated DNA sequencer at the University of Chicago Cancer Center DNA Sequencing Facility (Chicago, Ill.). Homology searches of GenBank databases were performed with the BLASTN and TBLASTN programs. Signal peptides were predicted with the SPScan program (Genetic Computer Group, Madison, Wis.). Transmembrane domains were predicted with the Swiss TMpred program (http://www.ch.embnet.org). Multiple sequence alignments were performed with the ClustalW program using MacVector (Genetic Computer Group, Madison, Wis.). Phylogenetic analysis to construct the best tree comprising amino acid sequences was performed by the uncorrected neighbor joining method using MacVector program (Genetic Computer Group, Madison, Wis.).

Analysis of mRNA Expression

Expression of the four PGRP mRNA transcripts was analyzed in 76 different human tissues using the Multiple Tissue Expression Array (Clontech). The Multiple Tissue Expression Array is a nylon membrane comprised of normalized amounts of poly-A⁺ RNA derived from 76 different human tissues and several control RNA and DNA samples immobilized in a matrix dot pattern. The following PGRP cDNA fragments, purified from agarose gels using the QIAquick PCR purification kit (Qiagen, Valencia, Calif.), were labeled with ³²P using the random primer labeling method (27) and purified on ChromaSpin columns (Clontech): for PGRP-L, nt 763 to 1459 (697 bp fragment, clone L11; SEQ ID NO: 21); for PGRP-Iα, nt 596 to 1019 (424 bp PCR fragment; SEQ ID NO: 24); for PGRP-Iβ, EST clone AI056693, corresponding to nt −53 to 418 (459 bp fragment); and for PGRP-S, EST clone AW076051, corresponding to nt 202 to 690 (489 bp fragment). The specific activity of the probes was 1.0-2.6×10⁶ dpm/ng. The membranes were hybridized overnight at 65° C. in ExpressHyb solution (Clontech), washed at high stringency as per the manufacturer's recommendation, and exposed to the Kodak X-Omat X-ray film with intensifying screens at −80° C. The membranes were subsequently stripped and re-hybridized with a positive control ubiquitin probe (Clontech) labeled as above. All of the probes were highly specific, as they did not cross-hybridize with other members of the PGRP family.

Expression of the four PGRPs in six tissues derived from the human immune system and twelve tissues derived from the human digestive system was evaluated and the sizes of different PGRP mRNA transcripts were estimated using Multiple Tissue Northern blots (Clontech) which have 2 μg of poly-A⁺ RNA per lane. ³²P-labeled PGRP cDNA fragments (the same as above, except that for PGRP-L, EST clone BE762960 was used) were hybridized to the membranes for 2 hrs at 68° C. in ExpressHyb solution (Clontech), washed at high stringency as recommended, and exposed to Kodak X-Omat X-ray film with intensifying screens at −80° C. The membranes were then stripped and re-hybridized with a positive control β-actin probe (Clontech) labeled as above.

Expression of the four PGRPs in 26 human tissues was also measured by PCR using Multiple Tissue cDNA panels containing normalized first-strand cDNA synthesized from DNA-free poly-A⁺ RNA (from Clontech). PCR amplifications were performed with Advantage 2 polymerase (Clontech) for 35 cycles under the conditions optimized for each set of primers, with the following primers: for PGRP-L, the L11 clone primers (see above); for PGRP-Iα, sense 5′ ATG ATG GCA GGG TGT ATG AAG G 3′ (SEQ ID NO: 17), and antisense, 5′ CTT GAA ATG AGG CCA GGT GCT GAT GA 3′ (SEQ ID NO: 18), which yield a 749 bp product; for PGRP-Iβ, the same primers as used for cloning, which yield a 1194 bp product; for PGRP-S, sense 5′ ATG TGG TGG TAT CGC ACA CG 3′ (SEQ ID NO: 19), antisense, 5′ GTC CTT TGA GCA CAT AGT TG 3′ (SEQ ID NO: 20), which yield a 342 bp product; and for human glyceraldehyde 3-phosphate dehydrogenase (GAPDH), used as a house-keeping gene control, the sense and antisense primers have been previously described (25), which yield a 452 bp product. PCR products were subjected to agarose gel electrophoresis and visualized by staining with ethidium bromide. The identity of all amplified PCR products was confirmed by probing of Southern blots with the same probes used for the Multiple Tissue Expression Arrays and by automated sequencing following extraction and purification of the bands from the agarose gel using a QIAquick PCR purification kit (Qiagen).

Expression of Recombinant PGRP Proteins

The four PGRPs and human CD4 (GenBank accession number M12807, a non-PGRP control) were subcloned from the pT-Adv vectors into the pcDNA3.1 mammalian expression vector (InVitrogen) and tagged at their C-terminal ends with the V5 and 6×His epitopes using TOPO directional cloning and Platinum Pfx polymerase (GIBCO/BRL Life Technologies, Rockville, Md.), as recommended by InVitrogen. The nucleotide sequences of all clones were confirmed by automated sequencing. Monkey kidney Cos-7 cells and human embryonic kidney HEK293 (ATCC), grown in DMEM medium with 10% fetal calf serum (28), were transfected with 0.4 μg/ml of PGRP or CD4 using lipofectamine, as previously described (28). The cells were lysed with 1% Triton-X100 (28) and the recombinant proteins were precipitated from the cell lysates with 2.5 μl of Ni-NTA-agarose (Qiagen), specific for the 6×His tag, as described (28). The Ni-NTA-bound proteins were separated on 11% PAGE gels and detected on Western blots with anti-V5 mouse monoclonal antibodies (mAbs; InVitrogen) and peroxidase-labeled anti-mouse IgG secondary antibodies (from Sigma, St Louis, Mo.), and enhanced chemiluminescence, as described (28).

Binding of PGRPs to PGN and Bacteria

Triton X-100 cell lysates (1 ml) from a 10 cm (Falcon 3003) plate of Cos-7 cells transiently transfected (as described above) with each PGRP or CD4 (a negative control that does not bind PGN) were incubated for 5 to 12 hrs at 4° C. on a rocking platform with 6.25 μl of control agarose or PGN-agarose (8, 25), or with 2.5 μl Ni-NTA-agarose (Qiagen). The agarose was sedimented by centrifugation at 10,000×g at 4° C., and washed three times with the cell lysis buffer (except for lysates from PGRP-Iβ-transfected cells, which were washed once). The agarose-bound proteins were released by boiling in a PAGE sample buffer containing 1% SDS and 1% 2-mercaptoethanol, separated on 11% PAGE gels, and detected on Western blots with anti-V5 mouse mabs and peroxidase-labeled anti-mouse IgG secondary antibodies, and enhanced chemiluminescence, as described (28).

Binding of PGRPs to bacteria (Bacillus subtilis, ATCC 6633, and Micrococcus luteus, ATCC 4698), or microgranular cellulose (a negative control; Sigma) was performed by incubating the cell lysates (as above) with 120 μg of bacteria or cellulose (used instead of the agarose), and then centrifuging the bacteria or cellulose and washing as described above for agarose (25). The bacteria-bound proteins were released and detected on Western blots as above.

Results

Cloning and Sequence Analysis of Three Novel Human PGRPs

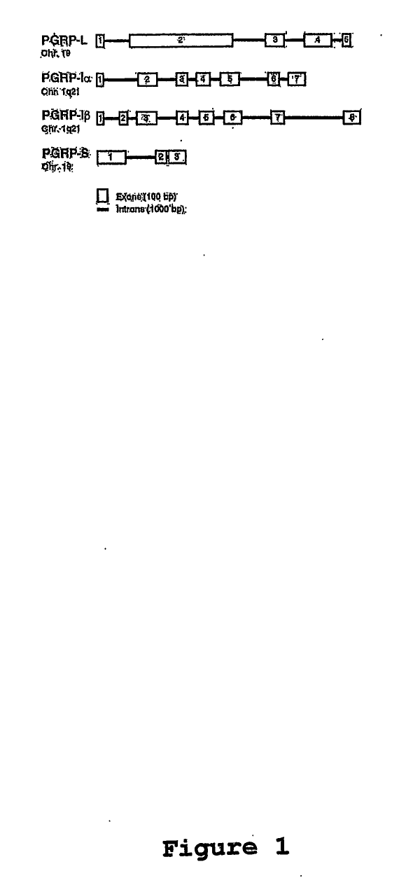

By searching GenBank databases for mammalian homologs of human PGRP, three novel human PGRP homologs have been identified as described herein. One was localized to chromosome 19, whereas the other two were localized to chromosome 1 (FIG. 1). Full length cDNA molecules encoding each of the PGRP genes were isolated using PCR and cDNA library screening. The first gene, which was designated PGRP-L (for PGRP-long, based on the nomenclature proposed for Drosophila PGRP with long transcripts; 26), was located on chromosome 19 and was comprised of five exons encoding a 576 amino acid protein.

The second and third genes were located on chromosome 1 (position q21) and encode 341 and 373 amino acid proteins, respectively, which have been designated PGRP-Iα and PGRP-Iβ (for PGRP-intermediate). This designation was based on their mutual homology and intermediate size compared to that of PGRP-L and the 196 amino acid original PGRP (23), which is now designated PGRP-S (for PGRP-short; 26). PGRP-Iα and PGRP-Iβ proteins were encoded by genes comprising 7 and 8 exons, respectively (FIG. 1). All PGRP-Iα and PGRP-Iβ exons were highly homologous, but PGRP-Iβ included an additional exon (exon 2) encoding protein sequence. A sequence homologous to the PGRP-Iβ exon 2 was also found in the PGRP-Iα gene; this sequence was not, however, expressed. PGRP-Iβ was 98% identical to the unpublished cDNA of Homo sapiens hypothetical protein SBBI67 (GenBank accession number AF242518). The PGRP-Iβ sequence described herein, however, differed from that of SBB167 since it comprised an additional twelve bp at the 5′ end of exon 3 and included eight other divergent nucleotides. These 12 bp were also absent from the EST clone AI056693, which spans PGRP-Iβ exons 1, 2, 3, and half of exon 4. Thus, exon 3 in PGRP-Iβ has an alternative splice site, that likely yields two alternatively spliced PGRP-Iβ isoforms.

The gene for the previously cloned PGRP-S (23) contains 3 exons (FIG. 1). The PGRP-S exons were located on chromosome 19, between nt 16,973 and 20,756 of the BAC clone 282485 (AC007785). Searches of the human genome did not reveal any other PGRP homologs and, therefore, the four PGRPs described herein likely constitute the entire human PGRP family.

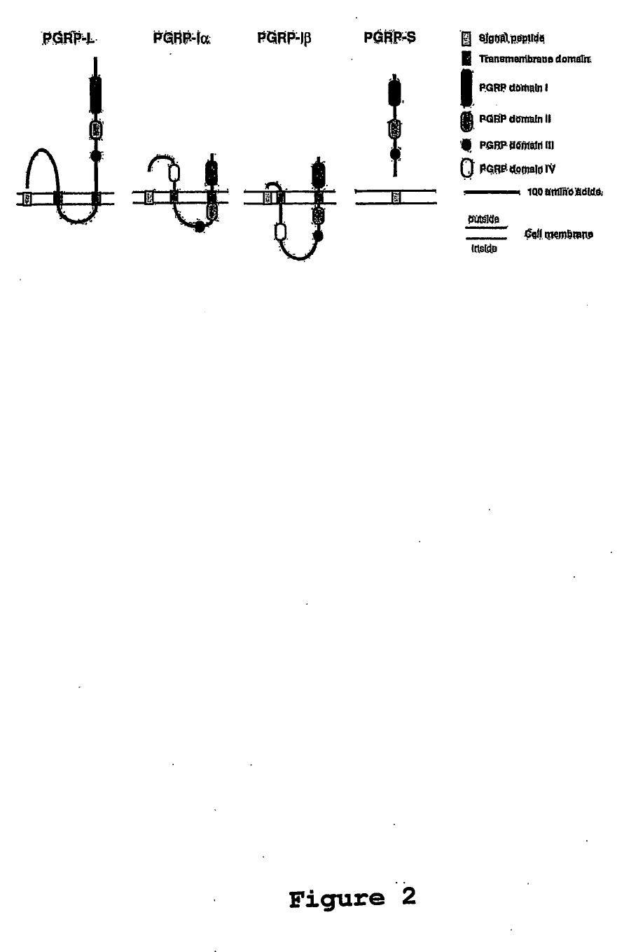

The C-terminal regions of all four human PGRPs were highly conserved and contained three PGRP domains (I, II, and III). These domains exhibited 54% to 69% conserved identity and 76% to 92% similarity (FIG. 2; data not shown). PGRP-Iα and PGRP-Iβ had an additional PGRP domain IV, located in the N-terminal halves of the molecules, which was 96% identical in PGRP-Iα and PGRP-Iβ, and was 64% identical (89% similar) to PGRP domain II (FIG. 2; data not shown).

The three PGRP domains (I, II, and III) were highly conserved in all of the 19 mammalian and insect PGRPs, for which full length clones have been isolated, and numerous residues or clusters of residues were fully conserved in virtually all of the above mammalian and insect PGRPs (data not shown). The identity and similarity conserved among mammalian and insect PGRP domains ranged from 47% to 57% in domain I, from 69% to 83% in domain II, and from 47% to 67% in domain III. Based on the presence and highly conserved nature of these PGRP domains, the three novel human PGRPs disclosed herein, together with PGRP-S, were classified as a new family of human PGRP molecules. Several other residues in the C-terminal region of all insect and mammalian PGRPs were also highly conserved, e.g., two cysteines (C419/425, C214/220, C246/252, and C67/73), arginine (R430, R225, R257, and R78), glutamine (Q433, Q228, Q260, and Q81) and histidine (H436, H231, Y263, and H84) residues located between PGRP domains II and III, or asparagine (N474, N269, N301, and N123), two glycines (G479/484, G274/279, G306/311, and G128/133), isoleucine (I480, I275, I307, and I129), phenylalanine (F482, F277, F309, F131), and proline (P491, P286, P318, and P140) residues located between PGRP domains I and II in PGRP-L, PGRP-Iα, PGRP-Iβ, and PGRP-S, respectively. Based on their conserved nature, the above amino acid residues were predicted to contribute to the tertiary structure, cellular location, and/or function of these PGRPs.

Regions that are most conserved in all insect and mammalian PGRPs are likely to be essential for the recognition of PGN and bacteria by these PGRP molecules. These regions correspond to PGRP domains I, II, III, and IV. Therefore, peptides corresponding to the entire PGRP domains I, II, III, and IV of human PGRP-L, PGRP-Iα, PGRP-Iβ, and PGRP-S (listed below in Table 2), or peptides corresponding to the most conserved fragments of these PGRP domains can be chemically synthesized or produced by recombinant DNA techniques. Methods for both of these approaches are well known to those of skill in the art

The remaining N-terminal portions of the PGRP molecules of the present invention exhibited very little homology within the PGRP family, except for a tryptophan residue (W337, W187, W219, and W39 in PGRP-L, PGRP-Iα, PGRP-Iβ, and PGRP-S), which was conserved in 18 out of 19 mammalian and insect PGRPs examined and five other residues having 74% to 89% conserved similarity. Thus, the total identities (similarities) among all human PGRPs were determined as follows: PGRP-L and PGRP-S, 40% (57%); PGRP-L and either PGRP-Iα or PGRP-Iβ, 33% and 32% (51% and 50%); PGRP-S and either PGRP-Iα or PGRP-Iβ, 43 and 42% (68% and 64%); and PGRP-Iα and PGRP-Iβ, 68% (80%).

All four human PGRPs had an N-terminal signal peptide (FIG. 2; data not shown). PGRP-L, PGRP-Iα and PGRP-Iβ also had two predicted transmembrane domains, and, therefore, were anticipated to be transmembrane proteins with two extracellular portions and one continuous cytoplasmic portion (FIG. 2). The locations of the transmembrane domains in each molecule were different, suggesting different organization of the molecules. In PGRP-L, all three PGRP domains were in one continuous extracellular portion. In PGRP-Iα, two extracellular portions comprised one PGRP domain each and the cytoplasmic portion comprised the remaining two PGRP domains. In PGRP-Iβ, only one extracellular portion had a PGRP domain I and the remaining three PGRP domains were located in the cytoplasmic portion (FIG. 2). PGRP-S did not have any transmembrane domains.

The locations of the signal peptides, transmembrane domains, and PGRP domains of the four human PGRPs are presented in Table 2.

TABLE 2
Amino acid residues corresponding to signal peptides,
transmembrane domains, and PGRP domains I, II, III, IV.

| | Signal | Transmembrane | PGRP domains | | | |
|---|---|---|---|---|---|---|
| PGRP | peptide | domains | I | II | III | IV |
| PGRP-L | 1-21 | 214-232 | 495-545 | 442-470 | 400-416 | |
| | | 325-343 | | | | |
| PGRP-Iα | 1-17 | 125-145 | 290-339 | 237-265 | 197-211 | 81-108 |
| | | 264-282 | | | | |
| PGRP-Iβ | 1-17 | 60-81 | 322-371 | 269-297 | 229-243 | 113-140 |
| | | 303-321 | | | | |
| PGRP-S | 1-21 | — | 144-193 | 90-118 | 50-64 | — |

The PGRP family described herein was not homologous to any other known gene family or to any known domains in other proteins. Moreover, apart from the conserved PGRP domains, the family members did not appear to comprise regions homologous to any other known motifs in either their cytoplasmic or extracellular portions. The apparent lack of homology suggested that PGRPs may have a unique function.

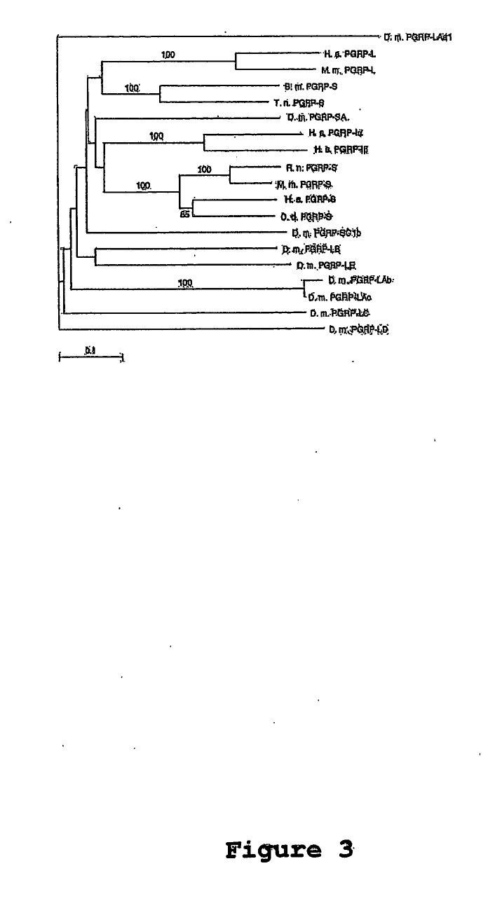

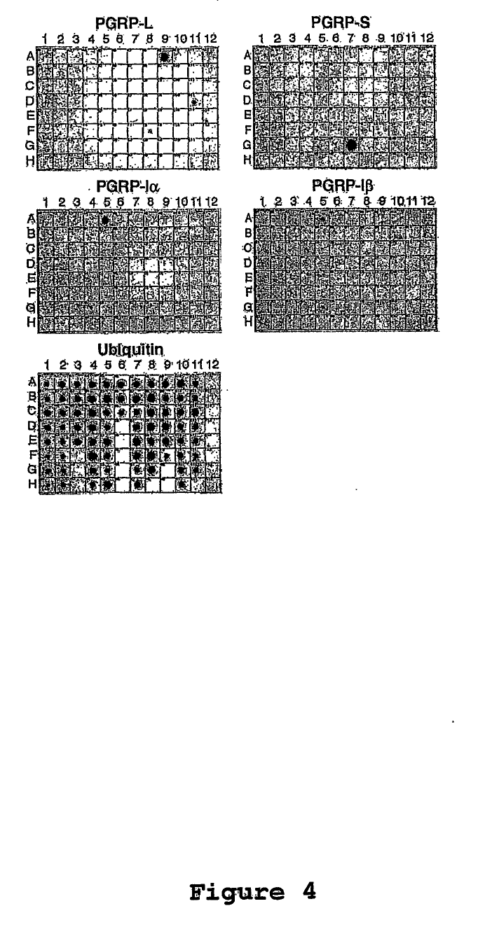

Phylogenetic analysis of all mammalian and insect PGRPs indicated that PGRP-S was the ancestral member of the PGRP family and PGRP-L evolved most recently, and confirmed that mammalian PGRP-S, human PGRP-I, and mammalian PGRP-L each form a separate, but closely linked branch (FIG. 3). This analysis also revealed that there were no insect homologs for either human PGRP-I and suggested that the mammalian PGRP-I branches were derived from a common ancestor of PGRP-S, following the divergent evolution of mammals (vertebrates) and insects. Mammalian PGRP-L form a separate branch that was apparently unrelated to the Drosophila PGRP-L branch, which suggested that mammalian PGRP-L did not originate from Drosophila PGRP-L, mammalian PGRP-S, or PGRP-I, but from a common ancestor of insect PGRP-S (FIG. 3).

Differential Expression of PGRP-L, PGRP-Iα, PGRP-Iβ, and PGRP-S

The expression pattern of mRNA transcripts encoding human PGRP-L, PGRP-Iα, PGRP-Iβ, and PGRP-S was evaluated in 76 human tissues and cells using Multiple Tissue Expression Array (FIG. 4). PGRP-L was strongly expressed in the adult liver and expressed at a tenth adult liver levels in fetal liver. Both PGRP-Iα and PGRP-Iβ were expressed predominantly in the esophagus, wherein expression levels of PGRP-Iα were 10 times higher than those of PGRP-Iβ. PGRP-S was very strongly expressed in the bone marrow, and expressed at 50 to 100 times lower levels in polymorphonuclear leukocytes and fetal liver. The overall expression was highest for PGRP-S, and approximately 10 times lower for PGRP-L, 100 times lower for PGRP-Iα, and 1000 times lower for PGRP-Iβ relative to PGRP-S, respectively (FIG. 4).

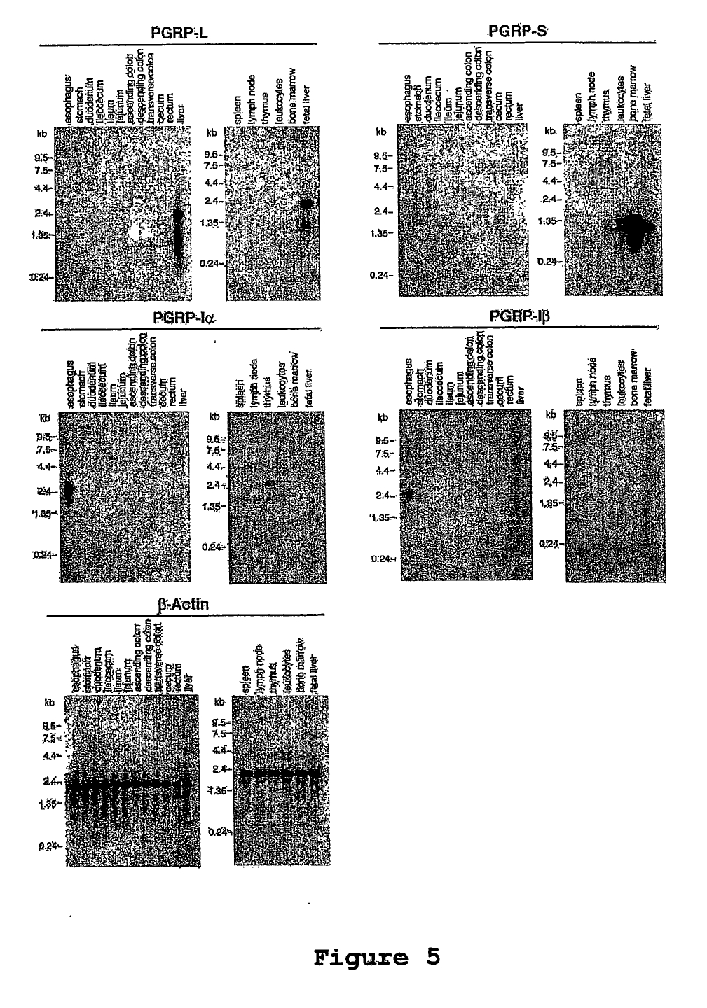

The expression of the above four PGRPs was also evaluated and the sizes of the PGRP-encoding mRNA transcripts determined by Northern blot analysis. For PGRP-L, 2.1 kb and 0.8 kb transcripts were detected in adult and fetal liver; for PGRP-Iα, a 2.8 kb transcript was detected in esophagus and thymus; for PGRP-Iβ, a 2.6 kb transcript was detected in esophagus; and for PGRP-S, 1.4 kb, 0.9 kb, and 0.5 kb transcripts were detected in bone marrow, a 0.9 kb transcript was detected in fetal liver, and 1.4 and 0.9 kb transcripts were detected in peripheral blood leukocytes (FIG. 5). The pattern of expression and the differences in the level of expression of these four PGRPs were similar in the Northern blot and expression array analyses.

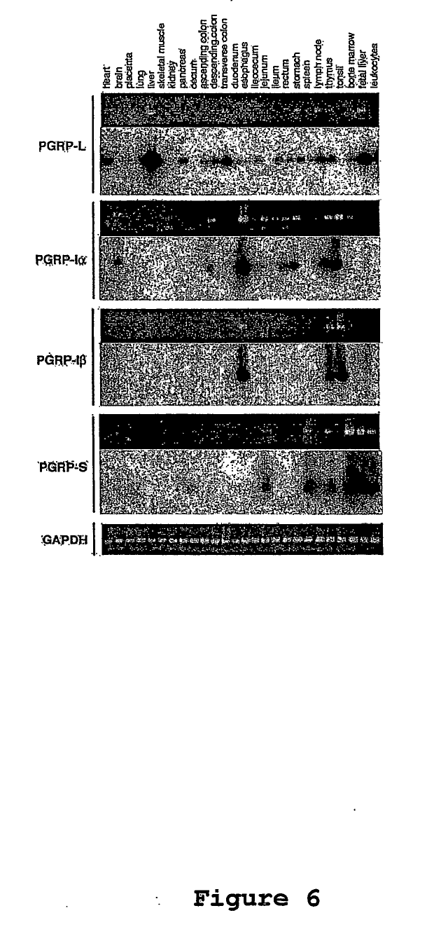

To determine if other tissues expressed low levels of these PGRPs, PCR amplification was performed on cDNA derived from 26 human tissues (FIG. 6). PGRP-L was the most widely expressed of all PGRPs. In addition to high expression in the liver and fetal liver, PGRP-L was also expressed to a much lower extent (100 to 1000 times less) in transverse colon, lymph nodes, heart, thymus, pancreas, descending colon, stomach, and testis (testis not shown in FIG. 6). In addition to the hereinabove identified high levels of expression observed in the esophagus, PGRP-Iα was also expressed in tonsils and thymus, and to a much lower extent in the stomach, descending colon, rectum, and brain. PGRP-Iβ was expressed only in the esophagus, tonsils and thymus. PGRP-S was highly expressed in the bone marrow, and to a lower extent in fetal liver and leukocytes. As previously determined, PGRP-S was expressed in human peripheral blood polymorphonuclear leukocytes, but not monocytes, lymphocytes, or NK cells (25). PGRP-S was also expressed at very low levels in spleen, jejunum, and thymus, but this low level of expression may have been due to contamination of these tissues with polymorphonuclear leukocytes, which may have also contributed to the low levels of PGRP-S expression previously observed in mouse spleen (25).

In summary, the human PGRP family members exhibited very selective and differential expression patterns. PGRP-S was highly expressed in the bone marrow, whereas PGRP-L was expressed predominantly in the liver, and PGRP-Iα and PGRP-Iβ were expressed predominantly in the esophagus.

All PGRP Proteins were Expressed and Bind to PGN and Bacteria

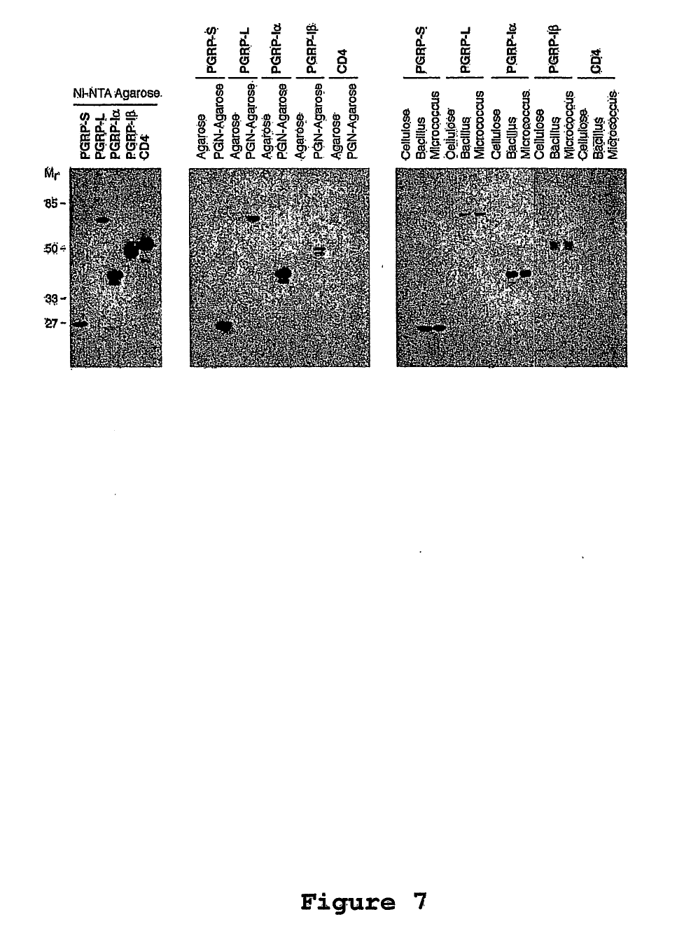

All four members of the human PGRP family were expressed following transient transfection of cDNA encoding each of these proteins into monkey (Cos-7) or human (HEK293, data not shown) cells. PGRP-L, PGRP-Iα, PGRP-Iβ, and PGRP-S were expressed as 65, 38, 46, and 24 kDa polypeptides, respectively, as detected on Western blots probed with anti-V5 tag mabs (FIG. 7). All four proteins were associated with the transfected cells, as predicted for transmembrane spanning proteins, and were completely solubilized by Triton X-100 containing lysis buffer. Unlike the other PGRPs examined, PGRP-S was also expressed as a secreted protein, as approximately half of the expressed PGRP-S could be detected in the culture supernatant (data not shown).

To determine the binding properties of expressed PGRP-L, PGRP-Iα, PGRP-Iβ, and PGRP-S, each was tested to evaluate its ability to recognize PGN and bacteria. All PGRPs bound to PGN-agarose but not to control agarose, whereas, a control unrelated transmembrane molecule, CD4, subcloned into the same vector and tagged with the same tags (V5 and 6×His), did not bind to either PGN-agarose or control agarose. All recombinant PGRPs and CD4 bound equally well to Ni-NTA-agarose as anticipated for 6×His tagged proteins (FIG. 7). These results demonstrated the specificity of PGRPs for PGN and confirmed that the binding to PGN was not due to the presence of either the V5 and 6×His tags in the recombinant molecules. All four PGRPs, but not CD4, also bound to the Gram-positive bacteria, Bacillus subtilis and Micrococcus luteus, but did not bind to microgranular cellulose (negative control) (FIG. 7). These results indicated that all four human PGRPs function to recognize PGN and PGN-containing Gram-positive bacteria. These results were consistent with previous results characterizing mouse PGRP-S (25), which bound PGN with nanomolar affinity and also bound to B. subtilis and M. luteus, the binding to all of which was shown to be independent of the C-terminal 6×His tag.

The binding of all PGRPs to PGN and bacteria was not equally strong. PGRP-S, PGRP-Iα, and PGRP-L showed strong binding to PGN and bacteria that did not diminish when PGN-agarose was extensively washed with a buffer containing 1 M NaCl and 1% Triton X-100, whereas the binding of PGRP-Iβ was much weaker and was substantially diminished after similar washing of PGN-agarose or bacteria. These results suggest that PGRP-S, PGRP-Iα, and PGRP-L bound to PGN with high affinity, while PGRP-Iβ bound to PGN with low affinity binding. Thus, PGRP-Iβ might have evolved to bind other as yet unidentified ligands or may require other molecules for high affinity binding.

In summary, the present invention provides nucleic acid sequences encoding full length open reading frames of three novel human pattern recognition molecules, PGRP-L, PGRP-Iα, and PGRP-Iβ (SEQ ID NOS: 1, 3, and 5, respectively). The present invention also provides amino acid sequences of full length PGRP-L, PGRP-Iα, and PGRP-Iβ (SEQ ID NOS: 2, 4, and 6, respectively). Together with the previously cloned PGRP-S (23), these four proteins can be classified into a new PGRP family of human pattern recognition molecules, based on the presence in all four proteins of three highly conserved PGRP domains, and also based on their ability to bind PGN, a ubiquitous component of bacterial cell walls.

The presence of PGRP domains and the ability to bind bacterial PGN and intact bacteria indicate that these PGRPs function in recognition of bacteria in innate immune responses. Human PGRP-S, PGRP-L, PGRP-Iα, and PGRP-Iβ are selectively expressed in different organs and have little homology outside the PGRP domains. This diversity suggests that binding of each mammalian PGRP to bacteria or PGN may produce a different biologic effect. Alternatively, binding of each mammalian PGRP to bacteria or PGN may produce similar biologic effects, but their expression in different tissues requires changes in the amino acid sequence to optimize expression levels. Alternatively, the tissue specific expression patterns of the different PGRPs may serve to circumscribe immune responses to sites of bacterial localization.

PGRP-L is primarily expressed in the liver. Human liver contains mainly parenchymal cells (˜80% of all liver cells are hepatocytes), and lower numbers of endothelial and Kupffer cells, that line blood vessels and sinusoids (29). Although liver is not generally considered a primary immune organ, liver participates in host defenses via hepatocyte production of acute phase proteins in response to infections and by clearing microorganisms from blood (29). The most prominent acute phase proteins include C-reactive protein, mannose-binding protein, serum amyloid A protein, α1-proteinase inhibitor, α1-acid glycoprotein, fibrinogen, α2-macroglobulin, and complement components (33, 34). Acute phase proteins are produced by liver parenchymal cells (hepatocytes) in response to infection, injury, or trauma. Moreover, it has been established that the HepG2/C3A human hepatoblastoma cell line (ATCC CRL-10741) expresses PGRP-L mRNA to the same extent as normal human unfractionated liver (C. Liu and R. Dziarski, unpublished). This cell line has many features of normal hepatocytes (parenchymal cells), including high production of albumin and many other liver-specific proteins, oxygen-dependent gluconeogenesis, nitrogen-metabolizing activity similar to perfused rat liver, and strong contact inhibition of growth. Thus, these results suggest that PGRP-L is expressed in liver parenchymal cells and may participate in recognition of bacteria by these cells.

PGRP-Iα and PGRP-Iβ are primarily expressed in the esophagus. Human esophagus is a 25 cm long hollow tubular passageway for the food from the oral cavity to the stomach. The esophagus is lined with thick stratified squamous epithelium that is incompletely keratinized (30, 31). The epithelium is surrounded by lamina propria with occasional lymphatic nodules, and by longitudinal and circular striated and smooth muscles. Mucosal glands are located only at both ends of the esophagus, and submucosal glands are located primarily in the upper half of the esophagus (30, 31). In view of the persistent exposure of the esophagus to microorganisms contained in food and the rarity of clinical diagnosis for bacterial infections of the esophagus, it must possess strong antimicrobial defenses. PGRP-Iα and PGRP-Iβ may participate in recognition of bacteria in the esophagus, and thus may play significant roles in esophageal antimicrobial defenses.

PGRP-Iα and PGRP-Iβ are also expressed (to a much lower extent) in tonsils and thymus, where they also may participate in recognition of bacteria. Moreover, their expression in the thymus suggests an intriguing potential role for these PGRPs in the maturation of T lymphocytes, which provides support for a heretofore unrecognized link between innate and acquired immunity.

PGRP-Iα and PGRP-Iβ, although highly homologous, have different transmembrane topology, i.e., PGRP-Iα has two extracellular and two intracellular PGRP domains, whereas, PGRP-Iβ has one extracellular and three intracellular PGRP domains (FIG. 2). Also, the binding affinity of PGRP-Iα for PGN and Gram-positive bacteria was much higher than that of PGRP-Iβ, which suggests that PGRP-Iβ might have evolved to recognize other ligands. Moreover, the expression of PGRP-Iα mRNA was 10 times higher than that of PGRP-Iβ mRNA. Therefore, despite high homology and expression in similar tissues, PGRP-Iα, and PGRP-Iβ may perform different functions.

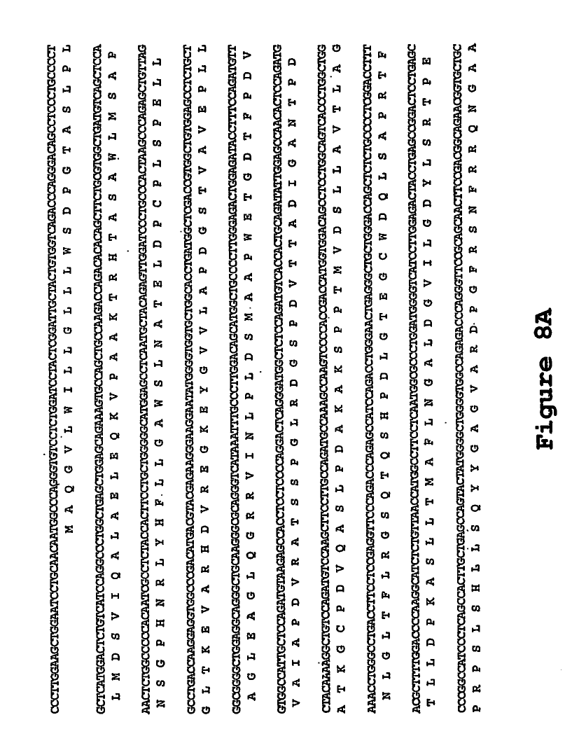

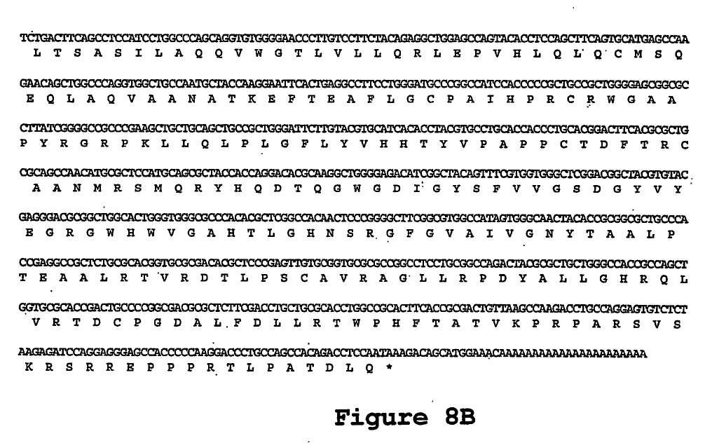

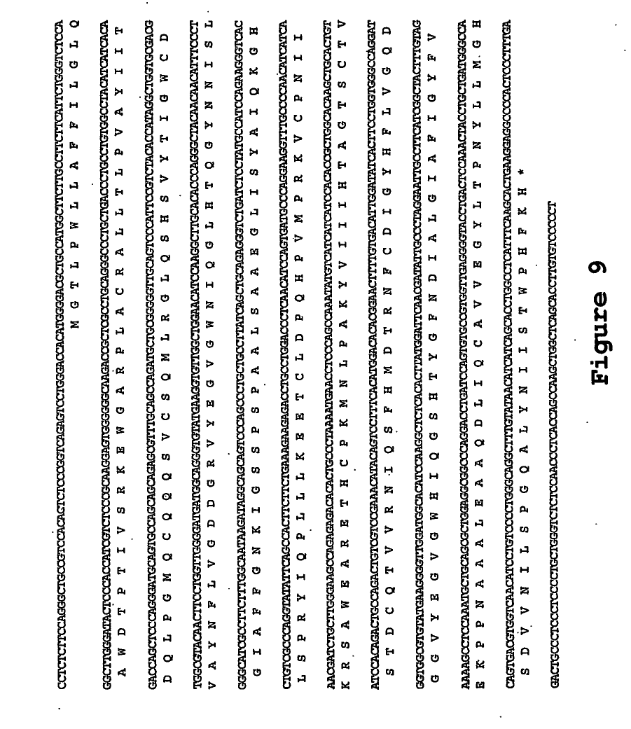

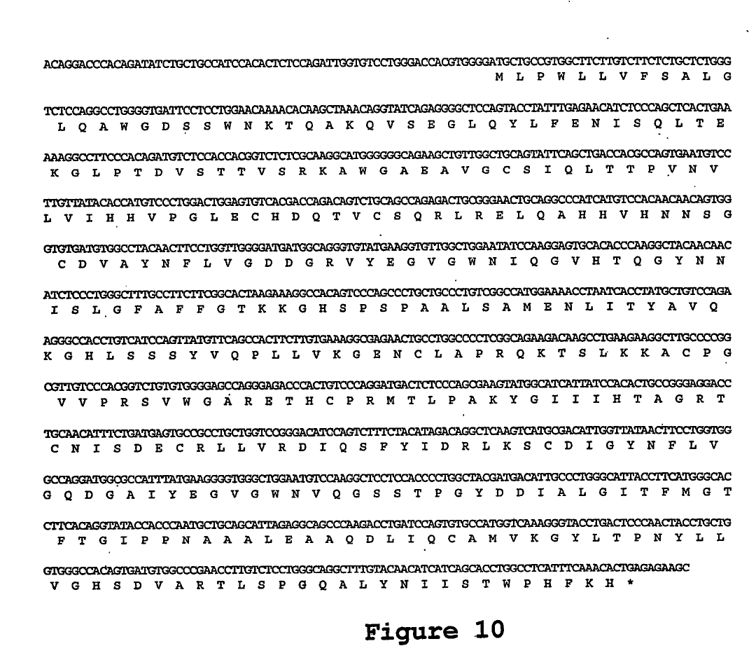

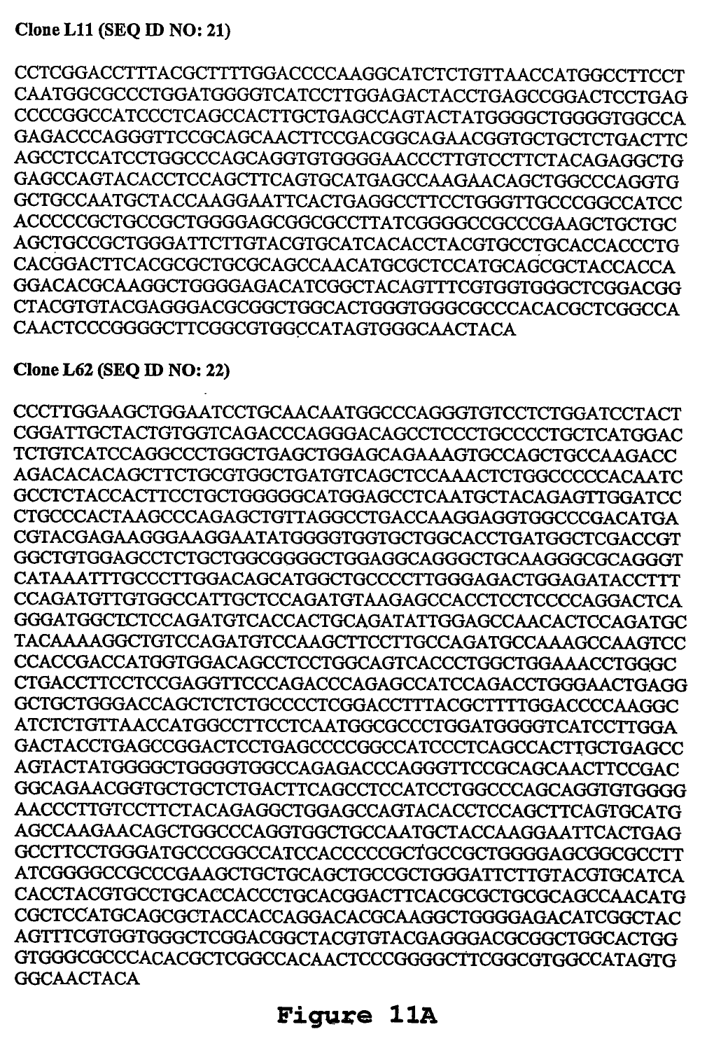

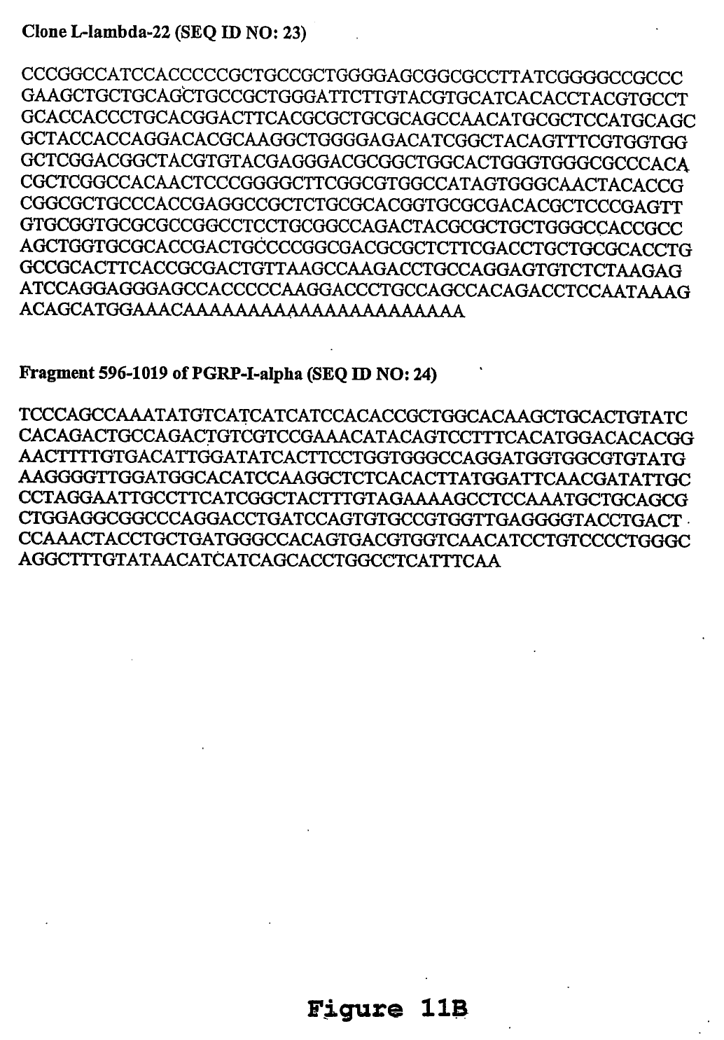

FIGS. 8, 9, and 10 show nucleic acid sequences (SEQ ID NOS: 1, 3, and 5) encoding amino acid sequences (SEQ ID NOS: 2, 4, and 6) corresponding to PGRP-L, PGRP-Iα and PGRP-Iβ, respectively. FIG. 11 shows the nucleic acid sequences of PGRP gene fragment/probes (SEQ ID NOS: 21, 22, 23, and 24) which may be used to advantage for a variety of purposes as described herein.

In summary, results presented herein demonstrate the existence of a novel family of pattern recognition molecules in humans that recognize bacterial cell wall PGN. The above family of PGRPs was conserved across millions of years of evolution, from insects to mammals. In mammals, PGRPs were found to be differentially expressed in bone marrow, liver, and esophagus, where they are likely to play a role in recognition of bacteria and activation of innate immune responses. See also reference 39, the entire contents of which is incorporated herein by reference. The present invention includes within its scope uses of PGRP nucleic acid and amino acid sequences described hereinabove in prophylactic treatment of patients at risk for bacterial infections and/or therapeutic treatment of patients suffering from localized or systemic bacterial infections. Moreover, agents capable of modulating the activity of PGRPs identified by methods of the present invention may also be of utility in a variety of clinical settings.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

References

1. Medzhitov, R., and Janeway, C. (2000) New Engl. J. Med. 343, 338-344

2. Hoffmann et al. (1999) Science 284, 1313-1318

3. Aderem, A., and Ulevitch, R. J. (2000) Nature 406, 782-787

4. Dziarski et al. (2000) Chemical Immunol. 74, 83-107

5. Dziarski et al. (2000) in Glycomicrobiology. (Doyle, R. J., ed) pp. 145-186, Kluwer Academic/Plenum Publishers, New York, N.Y.

6. Weidemann et al. (1994) Infect. Immun. 62, 4709-4715

7. Gupta et al. (1996) J. Biol. Chem. 271, 23310-23316

8. Dziarski et al. (1998) J. Biol. Chem. 273, 8680-8690

9. Schwandner et al. (1999) J. Biol. Chem. 274, 17406-17409

10. Yoshimura et al. (1999) J. Immunol. 163, 1-5

11. Takeuchi et al. (1999) Immunity 11, 443-451

12. Poltorak et al. (1998) Science 282, 2085-2088

13. Hemmi et al. (2000) Nature 408, 740-745

14. Du et al. (2000) Europ. Cytokine Network 11, 362-371

15. Schleifer, K. H., and Kandler, O. (1972) Bacteriol. Rev. 36, 407-477

16. Doyle, R. J., and Dziarski, R. (2001) in Molecular Medical Microbiology (Sussman, M., ed) pp. 137-153, Academic Press, London

17. Dunn et al. (1985) Dev. Comp. Immunol. 9, 559-568

18. Ashida, M., and Brey, P. T. (1997) in Molecular Mechanisms of Immune Responses in Insects (Brey, P. T., and Hultmark, D., eds) pp. 135-172, Chapman & Hall, London

19. Gupta et al. (1995) J. Immunol. 155, 2620-2630

20. Wang et al. (2000) J. Biol. Chem. 275, 20260-20267

21. Dziarski, R. (1980) J. Immunol. 125, 2478-2483

22. Yoshida et al. (1996) J. Biol. Chem. 271, 13854-13860

23. Kang et al. (1998) Proc. Natl. Acad. Sci. USA 95, 10078-10082

24. Ochiai, M., and Ashida, M. (1999) J. Biol. Chem. 274, 11854-11858

25. Liu et al. (2000) J. Biol. Chem. 275, 24490-24499

26. Werner et al. (2000) Proc. Natl. Acad. Sci. USA 97, 13772-13777

27. Sambrook, J., and Russell, D. W. (2001) Molecular Cloning, Cold Spring Harbor Lab. Press, Cold Spring Harbor, N.Y.

28. Dziarski et al. (2001) J. Immunol. 166, 1938-1944

29. Jones, A. L., and Spring-Mills, E. (1992) in Cell and Tissue Biology, 6th Ed. (Weiss, L., ed) pp. 685-714, Urban & Schwarzenberg, Baltimore, Md.

30. McDonald, G. B (1989) In Gastrointestinal Disease: Pathophysiology, Diagnosis, Management, 4th Ed. (Sleisenger, M. H., and Fordtran, J. S., eds) pp. 640-656, W. B. Saunders, Philadelphia, Pa.

31. Goyal, R. K. (2001) in Harrison's Principles of Internal Medicine, 15th Ed. (Braunwald, E., ed) pp. 1642-1649, McGraw-Hill, New York, N.Y.

32. Buckley, R. H. (1999) in Fundamental Immunology, 4^{th} Ed. (Paul, W. E., ed) pp. 1427-1453; Lippincott-Raven Publ., Philadelphia, Pa.

33. Janeway et al. (1999) in Immunobiology, 4^{th} Ed., pp. 375-390, Current Biology, London.

34. Sell, S. (1996) in Immunology, Immunopathology, and Immunity, Appleton & Lange, Stanford, Calif.

35. Michel et al. (2001) Nature 414, 756-759.

36. Gottar et al. (2002) Nature 416, 460-464.

37. Choe et al. (2002) Science 269, 359-362.

38. Ramet et al. (2002) Nature 416, 644-648.

39. Liu et al. (2001) J. Biol. Chem. 276, 34686-34693.

What is claimed is:

1. An isolated nucleic acid molecule having the sequence of SEQ ID NO: 1, said nucleic acid molecule comprising a nucleotide sequence encoding a PGRP-L about 576 amino acids in length, said encoded peptidoglycan recognition protein (PGRP) comprising a multi-domain structure including an N-terminal signal peptide, two predicted transmembrane domains, and three PGRP domains located in the extracellular portion.

2. The nucleic acid molecule of claim 1, which is DNA.

3. The DNA molecule of claim 2, which is a cDNA comprising a sequence approximately 1794 kilobase pairs in length that encodes said PGRP-L.

4. The DNA molecule of claim 2, which is a gene comprising introns and exons, the exons of said gene specifically hybridizing with the nucleic acid of SEQ ID NO: 1, and said exons encoding said PGRP-L.

5. An isolated RNA molecule transcribed from the nucleic acid of claim 1.

6. The nucleic acid molecule of claim 1, wherein said sequence encodes a PGRP-L having an amino acid sequence selected from the group consisting of SEQ ID NO: 2 and amino acid sequences encoded by natural allelic variants of said sequence.

7. The nucleic acid molecule of claim 6, which comprises SEQ ID NO: 1.

8. An antibody immunologically specific for a protein encoded by the nucleic acid of claim 1.

9. An antibody as claimed in claim 8, said antibody being monoclonal.

10. An antibody as claimed in claim 8, said antibody being polyclonal.

11. An isolated nucleic acid molecule having the sequence of SEQ ID NO: 3, said nucleic acid molecule comprising a sequence encoding a PGRP-Iα about 341 amino acids in length, said peptidoglycan recognition protein having a multi-domain structure including an N-terminal signal peptide, two predicted transmembrane domains, and four PGRP domains, two of said PGRP domains located on different extracellular portions and two of said PGRP domains located on the cytoplasmic portion.

12. The nucleic acid molecule of claim 11, which is DNA.

13. The DNA molecule of claim 12, which is a cDNA comprising a sequence approximately 1173 kilobase pairs in length that encodes said PGRP-Iα.

14. The DNA molecule of claim 12, which is a gene comprising introns and exons, the exons of said gene specifically hybridizing with the nucleic acid of SEQ ID NO: 3, and said exons encoding said PGRP-Iα.

15. An isolated RNA molecule transcribed from the nucleic acid of claim 11.

16. The nucleic acid molecule of claim 11, wherein said sequence encodes a PGRP-Iα having an amino acid sequence selected from the group consisting of SEQ ID NO: 4 and amino acid sequences encoded by natural allelic variants of said sequence.

17. The nucleic acid molecule of claim 11, which comprises SEQ ID NO: 3.

18. An antibody immunologically specific for a protein encoded by the nucleic acid of claim 11.

19. An antibody as claimed in claim 18, said antibody being monoclonal.

20. An antibody as claimed in claim 18, said antibody being polyclonal.

21. An oligonucleotide between about 10 and about 200 nucleotides in length, which specifically hybridizes with a protein translation initiation site in a nucleotide sequence encoding amino acids of SEQ ID NO: 2.

22. An oligonucleotide between about 10 and about 200 nucleotides in length, which specifically hybridizes with a protein translation initiation site in a nucleotide sequence encoding amino acids of SEQ ID NO: 4.

23. An isolated nucleic acid molecule having the sequence of SEQ ID NO: 5, said nucleic acid molecule comprising a sequence encoding a PGRP-Iβ about 373 amino acids in length, said peptidoglycan recognition protein having a multi-domain structure including an N-terminal signal peptide, two predicted transmembrane domains, and four PGRP domains, one of said PGRP domains located on an extracellular portion and three of said PGRP domains located on the cytoplasmic portion.

24. The nucleic acid molecule of claim 23, which is DNA.

25. The DNA molecule of claim 24, which is a cDNA comprising a sequence approximately 1194 kilobase pairs in length that encodes said PGRP-Iβ.

26. The DNA molecule of claim 24, which is a gene comprising introns and exons, the exons of said gene specifically hybridizing with the nucleic acid of SEQ ID NO 5, and said exons encoding said PGRP-Iβ.

27. An isolated RNA molecule transcribed from the nucleic acid of claim 23.

28. The nucleic acid molecule of claim 23, wherein said sequence encodes a PGRP-Iβ having an amino acid sequence selected from the group consisting of SEQ ID NO 6 and amino acid sequences encoded by natural allelic variants of said sequence.

29. The nucleic acid molecule of claim 23, which comprises SEQ ID NO: 5.

30. An antibody immunologically specific for a protein encoded by the nucleic acid of claim 23.

31. An antibody as claimed in claim 30, said antibody being monoclonal.

32. An antibody as claimed in claim 30, said antibody being polyclonal.

33. An oligonucleotide between about 10 and about 200 nucleotides in length, which specifically hybridizes with a protein translation initiation site in a nucleotide sequence encoding amino acids of SEQ ID NO: 6.

34. A plasmid comprising a nucleotide sequence selected from the group consisting of SEQ ID NO: 1, SEQ ID NO: 3, and SEQ ID NO: 5.

35. A vector comprising a nucleotide sequence selected from the group consisting of SEQ ID NO: 1, SEQ ID NO: 3, and SEQ ID NO: 5.

36. A retroviral vector comprising a nucleotide sequence selected from the group consisting of SEQ ID NO: 1, SEQ ID NO: 3, and SEQ ID NO: 5.

37. A host cell comprising at least one nucleic acid molecule having a sequence selected from the group consisting of SEQ ID NO: 1, SEQ ID NO: 3, and SEQ ID NO:5.

38. A host cell as claimed in claim 37, wherein said host cell is selected from the group consisting of bacterial, fungal, mammalian, insect and plant cells.

39. A host cell as claimed in claim 37, wherein said nucleic acid is provided in a plasmid and is operably linked to mammalian regulatory elements which confer high expression and stability of mRNA transcribed from said nucleic acid.

40. A host cell as claimed in claim 37, wherein said nucleic acid is provided in a plasmid and is operably linked to mammalian regulatory control elements in reverse anti-sense orientation.

41. A host animal comprising at least one nucleic acid molecule selected from the group consisting of SEQ ID NO: 1, SEQ ID NO: 3, and SEQ ID NO: 5.

42. A host animal as claimed in claim 41, wherein said animal harbors a homozygous null mutation in its endogenous PGRP gene wherein said mutation has been introduced into said mouse or an ancestor of said mouse via homologous recombination in embryonic stem cells, and further wherein said mouse does not express a functional mouse GPRP.

43. The transgenic mouse of claim 42, wherein said mouse is fertile and transmits said null mutation to its offspring.

44. The transgenic mouse of claim 42, wherein said null mutation has been introduced into an ancestor of said mouse at an embryonic stage following microinjection of embryonic stem cells into a mouse blastocyt.

45. A method for screening a test compound for inhibition of a PGRP mediated immune response, comprising:
a) providing a host cell expressing at least one PGRP-encoding nucleic acid having a sequence selected from the group consisting of SEQ ID NOS: 1, 3, and 5;
b) contacting said host cell with a compound suspected of inhibiting PGRP-mediated peptidoglycan binding activity; and
c) assessing inhibition of peptidoglycan binding mediated by said compound.

46. A method as claimed in claim 45, wherein inhibition of PGRP mediated peptidoglycan binding is indicated by restoration of a normal immune response.

47. A method as claimed in claim 46, wherein said inhibition of PGRP mediated peptidoglycan binding is indicated by a reduction of an immune response, comprising at least a reduction of inflammatory mediator production.

48. A composition comprising at least one peptidoglycan recognition protein in a pharmaceutically acceptable carrier, wherein said peptidoglycan recognition protein is selected from the group consisting of SEQ ID NOS: 2, 4, and 6, and functional fragments and derivatives thereof.

49. A kit for detecting the presence of PGRP encoding nucleic acids in a sample, comprising:
a) oligonucleotide primers specific for amplification of PGRP encoding nucleic acids;
b) polymerase enzyme;
c) amplification buffer; and
d) PGRP specific DNA for use as a positive control.
